(19) United States
(12) Patent Application Publication
(10) Pub. No.: US 2020/0208136 A1
(43) Pub. Date: Jul. 2, 2020

(54) METHOD OF ISOLATING NUCLEIC ACID

(71) Applicant: XING TECHNOLOGIES PTY LTD, Sinnamon Park, Queensland (AU)

(72) Inventors: Eugene J. H. Wee, Singapore (SG); Will Anderson, Brisbane (AU); Yadveer Singh Grewal, Brisbane (AU)

(21) Appl. No.: 16/633,731

(22) PCT Filed: Jul. 25, 2018
(86) PCT No.: PCT/AU2018/050770

§ 371 Date: Jan. 24, 2020

(30) Foreign Application Priority Data
Jul. 27, 2017 (AU) 2017902950

(51) Int. Cl. C12N 15/10; C12Q 1/6806

(52) U.S. Cl. CPC C12N 15/1006; C12Q 1/6806

(57) ABSTRACT

Disclosed herein is a method of isolating nucleic acid from a sample containing nucleic acid, the method comprising (a) exposing the sample to a thermoplastic polymer substrate under conditions that allow nucleic acid in the sample to reversibly bind to the substrate; (b) washing the nucleic acid-bound substrate of (a) under conditions that preferentially remove non-nucleic acid impurities bound to the substrate; and (c) exposing the washed nucleic acid-bound substrate of (b) to an elution buffer, thereby recovering the nucleic acid from the substrate.

### TECHNICAL FIELD

The present invention relates generally to a method of isolating nucleic acid, in particular, a method of isolating nucleic acid from biological material such as cell lysates.

### BACKGROUND

Nucleic acid (NA) isolation is non-trivial, as the performance of any DNA/RNA assay is dependent on the quality of the NA input.⁸ In point-of-care (POC) applications, the NA isolation processes is further complicated due to restrictions in availability of on-site resources. For instance, most routine lab-based NA isolation protocols (based on the Boom method⁹) typically require the use of centrifuges (for example, silica spin column-based methods.^{9,10}). However, in remote locales or home-based NA assays, access to centrifuges may not be possible. For this reason, various strategies have been proposed to circumvent such resource limitations. One example is the solid phase reversible immobilization (SPRI)¹¹ method which has recently gained popularity.¹¹⁻¹⁵ SPRI is typically based on the precipitation of NA onto surfaces (e.g. microparticles) which can then be resuspended in a compatible buffer after an alcohol wash. SPRI requires minimal equipment and hence is more suited for POC applications. However, conventional SPRI is limited by the need for multiple sample/liquid manipulation. Various strategies have since been developed to automate SPRI,¹⁶ but most POC-tailored approaches still require some form of micro-equipment^{13,17-19} which may not be suitable for low resource settings. Other contemporary NA isolation approaches²⁰ include using electric pulses to manipulate cellular lysis, NA isolation and concentration. However, these approaches are still largely proof-of-concept and/or require very specialized equipment.

In some iterations of SPRI, various forms of microparticle surface modification have been explored. These include carboxylic acid,¹¹ cellulose,^{14,15} silica (an extension of the Boom method) and chitosan.¹³ In general, biocompatible substrates with positive charged (cationic) functional groups are useful for NA isolation. In addition, some strategies to functionalize plastic surfaces with DNA include non-specific physisorption.²¹ However, physisorption of NA is usually counterproductive in NA assays as it reduces the availability of a target for bioanalysis.

The present disclosure solves, or at least partly alleviates, these problems by providing a simpler approach to solid phase reversible immobilization of nucleic acid that is compatible with low resource and/or POC applications.

### SUMMARY OF THE INVENTION

In an aspect disclosed herein, there is provided a method of isolating nucleic acid from a sample containing nucleic acid, the method comprising:
(a) exposing the sample to a thermoplastic polymer substrate under conditions that allow nucleic acid in the sample to reversibly bind to the substrate,
(b) washing the nucleic acid-bound substrate of (a) under conditions that preferentially remove non-nucleic acid impurities bound to the substrate; and
(c) exposing the washed nucleic acid-bound substrate of (b) to an elution buffer, thereby recovering the nucleic acid from the substrate;
   wherein the thermoplastic polymer substrate has a net negative charge in solution.

In another aspect, there is provided a composition comprising nucleic acid recovered by the methods disclosed herein.

In another aspect, there is provided a kit for isolating nucleic acid from a sample containing nucleic acid, the kit comprising:

(a) a thermoplastic polymer substrate, as herein described;
(b) an elution buffer, as herein described; and
(c) optionally, a cell lysis buffer, as herein described;
wherein the thermoplastic polymer substrate has a net negative charge in solution.

In another aspect, there is provided a thermoplastic polymer substrate, as herein described, for isolating nucleic acid from a sample containing nucleic acid in accordance with the methods disclosed herein, wherein the thermoplastic polymer substrate has a net negative charge when exposed to the sample.

### BRIEF DESCRIPTION OF THE FIGURES

FIG. 1A shows a general method and steps for nucleic acid (DNA) isolation from lysate material using a PLA-based, 3D printed DipStix. FIG. 1B is a gel image of DNA amplification products (amplicons) prepared by isothermal recombinase polymerase amplification (RPA) of HeLa cell genomic DNA (LINE1 target sequence), showing that different thermoplastic 3D printing substrates are capable of isolating DNA from lysate material for subsequent DNA amplification. The type of thermoplastic 3D printing substrate used and its manufacturer are provided along the top of panel B. (+): positive samples. (−): no DNA controls. DNA band sizes are as indicated.

FIG. 2 shows the performance and applications of DipStix in complex samples. (A) qPCR Ct values as a function of DNA concentration using BRAF and LINE1 primers. BRAF amplicons (DNA amplification products) are represented by the top line, whereas LINE1 amplicons are represented by the bottom line; error bars indicate SD (n=2). (B) Reproducible qPCR Ct values of DNA isolated from 7 independent DipStix with 10 ng/μL call lysates; error bars indicate SD (n=7). (C) Gel image of LINE1 and BRAF sequences amplified by RPA from crude cell lysates. (D) RT-qPCR profiles of miRNA and mRNA targets from crude cell lysates compared to Trizol extracted total RNA, from left to right: miR200a, miR15a, RNU6b, Actin and Her3. (E) Gel image of LINE1 sequences amplified by RPA from crude lysates of cells from cheek swabs and whole blood (NoT=No target control). (F) Gel image comparing PCR performance between DipStix isolated DNA and 1 μL of crude plant lysate added directly to the PCR reaction. Arabidopsis and tomato leaves were used.

FIG. 3A shows a representative gel image of RPA amplicons generated after the Dipstix were exposed to the DNA-containing lysis buffer for a period of time from 1 second to 5 minutes. FIG. 3B shows the average Ct values of RPA amplicons generated after the Dipstix were exposed to the DNA-containing lysis buffer for a period of 5 minutes, wash in water for five 1 second dips and eluted into PCR buffer for a period of time from a rapid (instantaneous) dip (t=−0) to 120 minutes (right panel); DNA standards of known amounts were included to estimate the amount of DNA available for PCR (see left panel). FIG. 3C shows the average Ct values of RPA amplicons generated after the Dipstix were exposed to the DNA-containing lysis buffer for 5 minutes, washed in either water (left bars) or PCR buffer (right bars) for a period of time from about 0.1 minutes to 60 minutes and subsequently eluted into PCR buffer for 5 minutes. FIG. 3D shows the average Ct values of RPA amplicons generated after the Dipstix were exposed to the DNA-containing lysis buffer containing increasing concentrations of guanidine chloride (GuHCl). FIG. 3E shows the effectiveness of DNA binding to the thermoplastic polymer substrate as a function of surface area, with representative gel images of RPA amplicons generated using DipStix printed to 1, 2, 3 mm diameters were submerged into the DNA-containing solution (left panel) and RPA amplicons generated when DipStix of an average diameter of 2 mm were submerged 5, 2.5 and 1.25 mm into the DNA-containing solution. FIG. 3F shows the average Ct values of RPA amplicons generated after increasing that print resolution of the thermoplastic polymer substrate up to 400 μm, noting that increasing the print resolution increases the surface area of the thermoplastic polymer substrate. FIG. 3G shows scanning electron microscope (SEM) images of Dichloromethane (DCM)-treated and untreated DipStix at 60× (top panel) and 5000× (bottom panel) magnification; scale bars are as shown. FIG. 3H is a magnified SEM image from the white box shown in FIG. 3G (see Untreated, 60× magnification; top panel) showing the structures between print layers. FIG. 3I shows a representative gel image of RPA amplicons of DNA isolated from a DNA-containing lysate using DCM-treated and untreated DipStix manufactured by two different 3D printers (Print1, Print2); DNA markers are shown on the far left and a negative control (No DNA) on the far right. FIG. 3J shows fluorescence imaging of Cy5-oligonucleotides (oligo) localizing between print layers of the thermoplastic polymer substrate. (H) FIG. 3K shows fluorescence imaging of the Dipstix during the DNA isolation steps: (i) after immersion into the DNA-containing lysis buffer with Cy5-oligonucleotides, (ii) after washes with water and (iii) after immersion into the PCR elution buffer. (I) FIG. 3L is a photograph of DipStix immersed in water (left) or lysis buffer (right) comprising a blue dye, showing wicking of the solutions along the DipStix.

FIG. 4 is a diagrammatical representation of a proposed mechanism by which nucleic acid may be binding to thermoplastic polymer substrates, showing association and dissociation constants (κ) of nucleic acid molecules (curly lines) and inhibitors (dots) as a result of changing buffer conditions during the extraction (isolation), wash and elution stages.

FIG. 5 shows the streaming (zeta, ζ) potential of several thermoplastic polymer substrates: beginning from the left and top to bottom: PLA Bilby 3D (natural), ABS Esun (white), Nylon Taulman 910, PLA ColorFab Copper and PLA ProtoPasta Conductive. The data show that all thermoplastic polymer substrates tested are negatively charged in a salt solution across a wide range of pH values. Measurements were performed in a solution of 1 mM NaCl on 1 mm films of the thermoplastic polymer substrate material using an Anton Paar SurPASS streaming potentiometer (Germany). pH titration was performed using solutions of 1 M NaOH. Measurements were analysed using the Fairbrother-Mastin approach.

FIG. 6 shows the quantitative PCR (qPCR) plot of amplicons generated from DNA isolated by Dipstix from a cell lysate comprising GuHCl and 100 ng of BT474-derived DNA (targeting LINE1 sequence). The cell lysate was prepared by exposing BT474 human breast cancer cells to the lysate buffer. The effect of washing the DipStix in water was examined to determine if the salt lysis buffer could be effectively removed from the DipStix so as to not interfere with the amplification of DNA by qPCR. These data demonstrate that washing was effective in removing any PCR-inhibiting quantities of GuHCl and that failing to wash results in the inhibition of PCR amplification.

FIG. 8 shows the melt analysis of amplicons generated from DNA isolated by Dipstix from the cell lysate comprising a lysis buffer of GuHCl and 100 ng of BT474-derived DNA. Amplification of DNA was optimal where the DNA was isolated from DipStix that were wash with water after being dipped into the DNA-containing lysis buffer and before being placed into the PCR amplification buffer.

FIG. 9 shows a qPCR plot of amplicons generated from DNA isolated by Dipstix from a cell lysate comprising Tris-HCl, EDTA, SDS, Proteinase K and 100 ng of BT474-derived DNA. The cell lysate was prepared by exposing BT474 human breast cancer cells to the lysis buffer. The effect of washing the DipStix in water was examined to determine if the Tris-based lysis buffer could be effectively removed from the DipStix so as to not interfere with the amplification of DNA by qPCR. The Tris-based lysis buffer appeared to inhibit PCR amplification of DNA, possibly attributed to the water wash being insufficient to remove SDS and Proteinase K, both of which are known to interfere with PCR amplification.

FIG. 10 shows the melt analysis of amplicons generated from DNA isolated by Dipstix from the cell lysate comprising a lysis buffer of Tris-HCl, EDTA, SDS, Proteinase K and 100 ng of BT474-derived DNA. The data show that no amplicons were generated in any of the samples, except for the positive control (250 ng DNA).

FIG. 11 shows the qPCR plot of amplicons generated from DNA isolated by Dipstix from a cell lysate comprising RIPA lysis buffer and 100 ng of BT474-derived DNA. The cell lysate was prepared by exposing BT474 human breast cancer cells to the RIPA buffer. The effect of washing the DipStix in water was examined to determine if the salt lysis buffer could be effectively removed from the DipStix so as to not interfere with the amplification of DNA by qPCR. These data demonstrate that washing was effective in removing any PCR-inhibiting quantities of RIPA buffer and that failing to wash results in the inhibition of PCR amplification.

FIG. 12 shows the melt analysis of amplicons generated from DNA isolated by Dipstix from the cell lysate comprising RIPA buffer and 100 ng of BT474-derived DNA. Amplification of DNA was optimal where the DNA was isolated from DipStix that were wash with water after being dipped into the DNA-containing lysis buffer and before being placed into the PCR amplification buffer. Non-washed samples resulted in different melt curves, which suggested washing is important to remove RIPA.

FIG. 13 shows the qPCR plot of amplicons generated from DNA isolated by Dipstix from a cell lysate comprising 100 ng of BT474-derived DNA and a lysis buffer of different GuHCl salt concentrations, ranging 375 mM to 6 M. These data demonstrate that lower salt concentrations worked better, noting that cycles times (CT) for DNA amplification decreased as the salt concentration was reduced. Higher salt concentrations (e.g., 6 M), still worked, but the CT values increased as a result. This increase in CT could negatively affect the lower limit of DNA amplification.

FIG. 14 shows the melt analysis of amplicons generated from DNA isolated by Dipstix from the cell lysate comprising 100 ng of BT474-derived DNA and a lysis buffer of different GuHCl salt concentrations, ranging from 6 M to 375 mM. Lower salt concentrations were found to be optimal for DNA amplification as compared to higher salt concentrations, although amplicons were produced at all salt concentrations. These data also show that washing the DipStix with water removes the inhibitory effect of the salt on qPCR amplification.

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

It is to be noted that, as used in the subject specification, the singular forms “a”, “an” and “the” include plural aspects unless the context clearly dictates otherwise. For example, reference to “a nucleic acid” includes a single nucleic acid molecule, as well as two or more nucleic acid molecules.

The present invention is predicated, at least in part, on the surprising finding that nucleic acid molecules are capable of reversibly binding to thermoplastic polymer substrates, such as those used for 3D printing, and that this property can be exploited for simple and rapid nucleic acid isolation that is compatible with downstream analyses, including the amplification and detection of target nucleic acid sequences.

Thus, in an aspect disclosed herein, there is provided a method of isolating nucleic acid from a sample containing nucleic acid, the method comprising:
(a) exposing the sample to a thermoplastic polymer substrate under conditions that allow nucleic acid in the sample to reversibly bind to the substrate;
(b) washing the nucleic acid-bound substrate of (a) under conditions that preferentially remove non-nucleic acid impurities bound to the substrate; and
(c) exposing the washed nucleic acid-bound substrate of (b) to an elution buffer, thereby recovering the nucleic acid from the substrate;
   wherein the thermoplastic polymer substrate has a net negative charge in solution.

### Thermoplastic Polymer Substrates

The term “thermoplastic polymer substrate” is understood to mean a thermosoftening plastic material that becomes pliable at above a specific temperature and solidifies upon cooling. Suitable thermoplastic polymer substrates will be familiar to persons skilled in the art, illustrative examples of which include those used for 3D printing, such as polyamides (e.g., nylon), polylactic acid (PLA), polystyrene (e.g., acrylonitrile butadiene styrene (ABS)) and composites or alloys thereof. Other illustrative examples of suitable thermoplastic polymer substrates include those used for injection moulding and vacuum forming.

Suitable thermoplastic polymer substrates are available commercially, illustrative examples of which include PLA Bilby 3D (natural), PLA ColorFab (white), ABS Esun (white), Nylon Taulman 910, PLA Bilby 3D Cherry Wood, PLA ColorFab Copper, PLA Bilby 3D Copper, PLA Bilby 3D Aluminium, PLA ProtoPasta Carbon Fibre and PLA ProtoPasta Conductive.

In an embodiment disclosed herein, the thermoplastic polymer substrate is selected from the group consisting of a polyamide, polylactic acid, acrylonitrile butadiene styrene and composites or alloys of any of the foregoing. Suitable composites and alloys of thermoplastic polymer substrates will be familiar to persons skilled in the art. In an embodiment, the composite or alloy comprises polylactic acid. In an embodiment, the thermoplastic polymer substrate is an alloy comprising polylactic acid and a metal. Suitable metals for use in thermoplastic polymer alloys will be familiar to persons skilled in the art, illustrative examples of which include copper, aluminium and titanium. In an embodiment, the metal is selected from the group consisting of copper and aluminium.

Suitable composites of thermoplastic polymer substrates will also be familiar to persons skilled in the art, illustrative examples of which include composites comprising polylactic acid and conductive material, such as carbon. In an embodiment, the thermoplastic polymer substrate is composite comprising polylactic acid and carbon.

As noted elsewhere herein, the thermoplastic polymer substrate will suitably have a net negative charge in solution. In an embodiment, the thermoplastic polymer substrate will display a negative streaming (zeta; ζ) potential across a range of pH values. In an embodiment, the thermoplastic polymer substrate will display a negative streaming potential across a pH range of about 5 to about 11. In an embodiment, the thermoplastic polymer substrate is characterised by a net negative charge when exposed to a salt solution. As noted elsewhere herein, the thermoplastic polymer substrate will suitably have a net negative charge when exposed to a solution of 1 mM NaCl. It is to be noted, however, that the property(ies) that make the thermoplastic polymer substrate suitable for use in accordance with the methods disclosed herein is not limited to exposure of the substrate to a nucleic acid-containing sample comprising 1 mM NaCl. As described elsewhere herein, the thermoplastic polymer substrates will be capable of isolating nucleic acid from a nucleic acid-containing solution that comprises a salt other than NaCl and a salt concentration other than 1 mM. In an embodiment, the thermoplastic polymer substrate will have a net negative charge in a solution comprising a chaotropic salt (e.g., guanidine HCl), as described elsewhere herein.

As noted elsewhere herein, thermoplastic polymers have the advantage of allowing substrates to be formed into almost any suitable size and shape. The size and shape of the thermoplastic polymer substrate will typically depend on the intended use. For instance, where the cell lysate is contained in a 0.5 mL Eppendorf tube, the substrate may be formed into an elongated shape having (i) a length that is able to extend into the tube and make contact with the cell lysate therein and (ii) an average diameter that is less than or equal to the diameter of the tube at its base such that the elongated substrate is capable of being inserted into the tube and make contact with the cell lysate material.

In an embodiment, the thermoplastic polymer substrate has an elongated structure with an average diameter from about 1 mm to about 3 mm. In an embodiment, the thermoplastic polymer substrate has an elongated structure with a length from about 1 to about 30 mm, preferably from about 10 mm to about 15 mm in length.

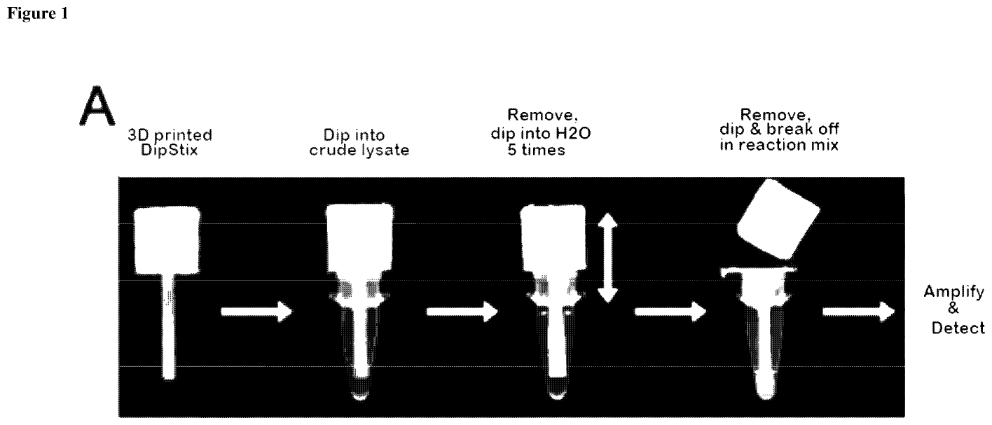

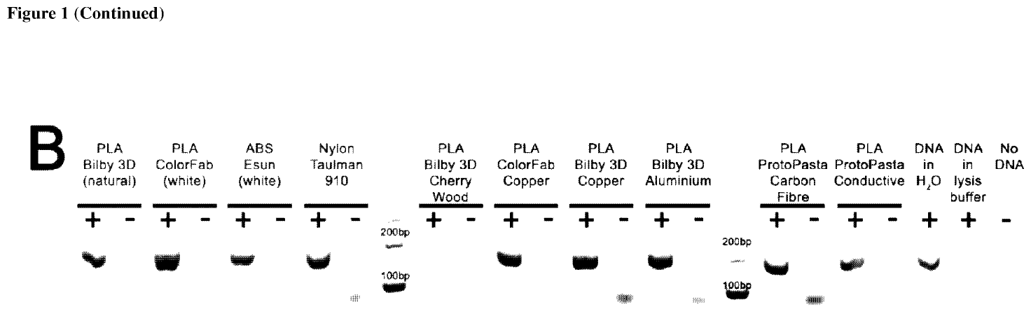

It is to be understood that the thermoplastic polymer substrate need not have a uniform elongated shape. For instance, as described elsewhere herein, the substrate may have a first portion and a second portion, wherein the first portion has an average diameter that is greater than the average diameter of the second potion. An illustrative example of a substrate having two different size portions is shown in FIG. 1 (also referred to herein as the “DipStix”).

In another embodiment, the thermoplastic polymer substrate has a substantially cylindrical structure or configuration, such as a channel or tube (e.g., a capillary tube). Such configuration may lend itself to microfluidic applications, whereby a nucleic acid-containing sample can be guided through the tube or channel in a microfluidic array, followed by a wash solution and then an elution buffer to recover the nucleic acid bound to the substrate. In another embodiment, the thermoplastic polymer substrate is configured as a tube and applied to the tip of a suction device, such as a syringe. The tubular substrate can be attached to the tip of the suction device or it can be integrally formed onto the tip of the device. The tubular substrate can then be inserted into a nucleic acid-containing sample and the sample drawn up through the tubular substrate by suction, whereby the nucleic acid in the sample binds to the inner surface of the tubular substrate. The tubular substrate can then be inserted into a wash solution, which is then drawn up through the tubular substrate by suction, thereby washing the substrate so as to remove non-nucleic acid impurities. The tubular substrate can then be inserted into an elution buffer, which is then drawn up through the tubular substrate by suction, thereby eluting the nucleic acid from the substrate and recovering the eluted nucleic acid into a collection chamber. Alternatively, the tubular substrate can be stored indefinitely after the wash step for subsequent elution of the bound nucleic acid. The thermoplastic polymer substrate can have a visibly smooth surface, or it may have an uneven or a textured surface, illustrative examples of which include a dimple pattern (as seen, e.g., on the surface of golf balls), a criss-cross pattern, a fish scale pattern and a palm scale pattern.

In another embodiment, the thermoplastic polymer substrate has a planar structure (e.g., a sheet) having a length and width that is greater than its thickness. The planer structure may be formed as a solid sheet or a sheet of woven strands of thermoplastic polymer material. In some embodiments, substrates formed of woven thermoplastic polymer strands have a pliable characteristic; that is, they retain a plasticity allowing them to be molded into one or more desirable shape. In some embodiments, the thermoplastic polymer substrate comprises a porous structure, for example, by incorporating pores that allow the passage of liquid. Alternatively, or in addition, the porous substrate comprises strands of thermoplastic polymer material that are woven to create a mesh-like structure. In an embodiment, a thermoplastic polymer substrate having a porous structure can be used as a filter. For example, a sample containing nucleic acid (e.g., a cell lysate), the wash and the elution buffer can be passed through the porous thermoplastic polymer substrate, whereby the nucleic acid in the sample binds to the substrate and is subsequently recovered by the elution buffer in accordance with the methods disclosed herein.

In another embodiment, the thermoplastic polymer substrate comprises one or more vessels for carrying a solution. Illustrative examples of suitable vessel include a tube and a well. In an embodiment, the thermoplastic polymer substrate has a multi-well configuration (e.g., a 96-well plate). Such configuration allows multiple samples to be processed in accordance with the methods described herein, either simultaneously or consecutively. Where the thermoplastic polymer substrate is a vessel, the nucleic acid-containing sample can be placed into the vessel for a period of time to allow the nucleic acid to bind to the substrate. The sample is then removed from the vessel (e.g., by suction) and the vessel washed so as to remove non-nucleic acid impurities from the substrate. An elution buffer can then be placed into the vessel, thereby eluting the nucleic acid from the substrate. The elution buffer containing the eluted nucleic acid can then be recovered from the vessel for subsequent storage or analysis (e.g., target nucleic acid amplification). Alternatively, the elution buffer can be kept in the vessel for storage and/or for subsequent analysis. For example, target nucleic acid amplification can be performed in the thermoplastic polymer vessel. This has an advantage of minimising the risk of cross-contamination where samples are transferred from one vessel to another.

### Sample Containing Nucleic Acid

As used herein, the term “sample” is understood to mean any solution comprising nucleic acid. The term “nucleic acid” is understood to mean ribonucleic acid (RNA) and deoxyribonucleic acid (DNA), illustrative examples of which include messenger RNA (mRNA), ribosomal RNA (rRNA), transfer RNA cDNA and genomic DNA, and include both eukaryotic and prokaryotic nucleic acid, mitochondrial DNA, chloroplast DNA (cpDNA), circulating free DNA (cfDNA) and circulating tumour DNA (ctDNA). The term “nucleic acid” also includes artificial nucleic acid analogues, peptide nucleic acids, morpholino- and locked nucleic acids, glycol nucleic acids, and threose nucleic acids, as distinguished from naturally-occurring nucleic acid, typically by modifications made to the backbone of the nucleic acid molecules.

In an embodiment, the sample is a biological sample. Illustrative examples of biological samples include blood, serum, plasma, urine, semen, amniotic fluid, bronchiolar lavage fluid (BAL), sputum and spinal fluid. The sample can be a naturally-occurring biological sample obtained from an organism (e.g., a prokaryote or a eukaryote) without further processing (e.g., urine, semen, spinal fluid, amniotic fluid), or it may be a biological sample obtained from an organism and undergone a processing step, such as purification to remove at least some impurities. In an embodiment, the sample contains less than 20%/by weight (w/w) nucleic acid. By “less than 20% w/w” is meant 19%, 18%, 17%, 16%, 15%, 14%, 13%, 12%, 11%, 10%, 9%, 8%, 7%, 6%, 5%, 4%, 3%, 2%, 1%, 0.5%, 0.1%, 0.05%, 0.01%, 0.005%, 0.001%, 0.0005%, 0.0001% and so on.

In an embodiment, the sample is a cell lysate. It is understood that cell lysates are formed by disrupting the cell and nuclear membranes of one or more cells to release the contents of the cell(s), in particular the nucleic acid content of the cell(s). Suitable methods of preparing a cell lysate will be familiar to persons skilled in the art, illustrative examples of which include osmotic shock lysis, lysis with chaotropic salts (e.g., GuHCl), enzymatic digestion, detergent lysis (e.g., non-ionic surfactants such as Triton X100) and mechanical homogenization. In an embodiment, the cell lysate is prepared by suspending the cell(s) in a lysis buffer. Suitable lysis buffers will be familiar to persons skilled in the art, illustrative examples of which include NP-40 lysis buffer, radio-immuno-precipitation assay (RIPA) lysis buffer and non-ionic surfactant-based lysis buffer.

In an embodiment, the sample comprises a chaotropic salt. Suitable chaotropic salts will be familiar to persons skilled in the art, illustrative examples of which include guanidine HCl, guanidine thiocyanate, urea and lithium perchlorate. In an embodiment, the chaotropic salt is guanidine chloride (GuHCl). As noted elsewhere herein, the thermoplastic polymer substrates were capable of binding and recovering nucleic acid from a sample containing nucleic acid across a range of lysis buffer salt concentrations. Thus, in an embodiment, the sample comprises a salt concentration that is from about 375 mM to about 6M (e.g., 375 mM, 400 mM, 500 mM, 600 mM, 700 mM, 800 mM, 900 mM, 1.0 M, 1.5 M, 2 M, 2.5 M, 3 M, 3.5 M, 4 M, 4.5 M, 5 M, 5.5 M and 6 M). In an embodiment, the sample comprises a salt concentration that is from about 375 mM to about 3M. In an embodiment, the sample comprises a salt concentration that is from about 375 mM to about 1.5M In an embodiment, the sample comprises a salt concentration that is about 1.5 M.

As noted elsewhere herein, the inventors have surprisingly found that nucleic acid will bind to thermoplastic polymer substrates almost instantaneously; for example, where the substrate is merely dipped into the solution containing nucleic acid for a period of no more than 1 second. Moreover, exposure for longer periods (e.g., up to 5 minutes) does not result in a discernible increase in the quantity of nucleic acid that is recovered from the solution. These data show that exposing the sample to the thermoplastic polymer substrate for a very brief period is sufficient to allow the nucleic acid in the sample to bind to the substrate for subsequent recovery. In an embodiment, step (a) comprises exposing the sample to the thermoplastic polymer substrate for a period of time from about 0.5 seconds to about 5 minutes, preferably for a period of time from about 0.5 seconds to about 1 minute, more preferably for a period of time from about 0.5 seconds to about 30 seconds, even more preferably for a period of time from about 0.5 seconds to about 1 second. In an embodiment, exposing the cell lysate to the thermoplastic polymer substrate comprises dipping the thermoplastic polymer substrate into the sample; for example, immersing at least a portion of the substrate into the sample and then immediately removing the substrate from the sample. In another embodiment, the sample containing nucleic acid (e.g., cell lysate) may be applied to the substrate; for example, by dripping the sample onto the substrate and allowing the sample to run off the surface of the substrate.

### Wash

As noted elsewhere herein, step (b) comprises washing the nucleic acid-bound substrate of (a) under conditions that preferentially remove non-nucleic acid impurities bound to the substrate. Suitable conditions that preferentially remove non-nucleic acid impurities bound to the solid substrate will be familiar to persons skilled in the art, illustrative examples of which include buffered solutions (e.g., Tris-buffered saline, phosphate buffered saline), salt solutions (e.g., NaCl, guanidine chloride) and, low EDTA TE buffer, 0.5% Tween solution, 0.5% triton solution, 70% ethanol and water. In some embodiments, the nucleic acid-bound substrate of (a) may be washed with the lysis buffer used to prepare a cell lysate to which the substrate is exposed in step (a).

In an embodiment, step (b) comprises washing the nucleic acid-bound substrate in water. As noted elsewhere herein, the inventors have surprisingly found that washing the nucleic acid-bound substrate in water for a period of between 0.1 to about 5 minutes was sufficient to remove undesirable, non-nucleic acid impurities, such as those that would otherwise have inhibited subsequent target sequence amplification. The inventors also surprisingly found that minimising the wash step with water to a period of less than about 5 minutes was optimal for nucleic acid recovery and subsequent target sequence amplification. Thus, in an embodiment, step (b) comprises washing the nucleic acid-bound substrate for a period from about 5 seconds to about 5 minutes. In another embodiment, step (b) comprises washing the nucleic acid-bound substrate for a period from about 5 seconds to about 1 minutes. In another embodiment, step (b) comprises dipping the nucleic acid-bound substrate into the wash solution; for example, immersing at least a portion of the nucleic acid-bound substrate into a wash solution (e.g., water) the solution and then immediately removing the substrate from the wash solution. In a preferred embodiment, step (b) comprises dipping the nucleic acid-bound substrate into a wash solution more than once, preferably from twice to about 5 times. In other embodiments, step (b) comprises dipping the nucleic acid-bound substrate consecutively into more than wash solution. For example, the nucleic acid-bound substrate is dipped into a first vessel comprising a wash solution, then dipped into a second vessel comprising the same or a different wash solution, then dipped into a third vessel comprising the same or a different wash solution than the wash solutions of the first and/or second vessels, and so on.

In another embodiment, the nucleic acid-bound substrate can be washed by applying the wash solution to the surface of the substrate to which the nucleic acid has bound; for example, by running a volume of wash solution (e.g., water) over the surface of the substrate to which the nucleic acid has bound.

### Elution

As used herein, the term “elution buffer” is understood to mean a solution that is capable of eluting (i.e., dissociating) the nucleic acid from the substrate to which they were bound after wash step (b). Suitable elution buffers will be familiar to persons skilled in the art, illustrative examples of which include PCR buffers and TE buffers. In an embodiment, the elution buffer is a PCR buffer. Suitable PCR buffers will be familiar to persons skilled in the art, an illustrative example of which includes the Kapa2G™ Buffer A or Kapa2G™ Buffer M (Sigma-Aldrich).

As noted elsewhere herein, the inventors have surprisingly found that nucleic acid can be eluted from the substrate to which they are almost instantaneously; for example, where the nucleic acid-bound substrate is merely dipped into the solution containing nucleic acid for a period of no more than 1 second. Moreover, exposure for longer periods (e.g., up to 120 minutes) does not result in a discernible increase in the quantity of nucleic acid that is recovered from the substrate. These data show that exposing the nucleic acid-bound substrate to an elution buffer for a very brief period of time is sufficient to allow the nucleic acid to be eluted from the substrate and recovered in the elution buffer.

In an embodiment, step (c) comprises exposing the washed nucleic acid-bound substrate to the elution buffer for a period of time from about 0.5 seconds to about 5 minutes, preferably for a period of time from about 0.5 seconds to about 1 minute, more preferably for a period of time from about 0.5 seconds to about 1 second. In an embodiment, exposing the washed nucleic acid-bound substrate to the elution buffer comprises dipping the washed nucleic acid-bound substrate into the elution buffer; for example, immersing at least a portion of the washed nucleic acid-bound substrate of (b) into the elution buffer and then immediately removing the substrate from the elution buffer.

In another embodiment, the nucleic acid can be eluted and recovered from the washed nucleic acid-bound substrate by applying the elution buffer onto the surface of the substrate to which the nucleic acid is bound; for example, by dripping a volume of the elution buffer onto the surface of the substrate to which the nucleic acid has bound and then collecting the elution buffer.

In an embodiment, the elution buffer comprises one or more components and/or reagents for performing nucleic acid amplification. Suitable components and/or reagents for performing nucleic acid amplification will be familiar to persons skilled in the art, illustrative examples of which include primers and/or probes that specifically hybridize to the target nucleic acid sequence of interest, enzymes suitable for amplifying nucleic acids, including various polymerases (e.g., Reverse Transcriptase, Taq, Sequenase™ DNA ligase etc. depending on the nucleic acid amplification technique employed), deoxynucleotides and buffers to provide the necessary reaction mixture for amplification and capture probes labelled with a detectable moiety (e.g., a fluorescent moiety). An advantage of an elution buffer comprising one or more components and/or reagents for performing nucleic acid amplification is that nucleic acid amplification can be performed immediately following elution of the nucleic acid and, hence, without the need to add further components and/or reagents to the elution buffer, thus minimising the risk of cross-contamination and non-specific nucleic acid amplification. An example of this approach is illustrated in FIG. 1.

In another aspect, there is provided a composition comprising nucleic acid recovered by the methods disclosed herein.

### Target Nucleic Acid Amplification

As described elsewhere herein, the inventors have surprisingly found that the thermoplastic polymer substrates are capable of reversibly binding nucleic acid molecules while allowing non-nucleic acid impurities, such as inhibitors of nucleic acid amplification, to be preferentially removed. As a result, the thermoplastic polymer substrate can be used to isolate nucleic acid molecules from non-nucleic acid impurities that may be present in a sample that would otherwise inhibit subsequent nucleic acid amplification and/or analysis. Thus, in an embodiment, the method described herein further comprises amplifying a target nucleic acid sequence from the nucleic acid recovered in step (c). Suitable methods for amplifying nucleic acid will be familiar to persons skilled in the art, illustrative examples of which include those disclosed in Green and Sambrook, (2012; “Molecular cloning: a laboratory manual”, fourth edition; Cold Spring Harbor, N. Y.) and Ausubel et al., (2003; “Current Protocols in Molecular Biology”; John Wiley & Sons, Inc). Where the target nucleic acid sequence is RNA, it may be desired to convert the RNA to a complementary DNA. In some embodiments, the nucleic acid is amplified by a template-dependent nucleic acid amplification technique. A number of template dependent processes are available to amplify a target sequence. An exemplary nucleic acid amplification technique is the polymerase chain reaction (PCR), which is described in detail in U.S. Pat. Nos. 4,683,195, 4,683,202 and 4,800,159, Ausubel et al. (supra), and in Innis et al., (“PCR Protocols”, Academic Press, Inc., San Diego Calif., 1990). A reverse transcriptase PCR amplification procedure may be performed in order to quantify the amount of mRNA amplified. Methods of reverse transcribing RNA into cDNA are well known and described in Sambrook et al., 2012, supra. Alternative methods for reverse transcription utilize thermostable, RNA-dependent DNA polymerases. These methods are described in WO 90/07641. Polymerase chain reaction methodologies are well known in the art.

In an embodiment, the template-dependent amplification involves quantification of transcripts in real-time. For example, RNA or DNA may be quantified using the Real-Time PCR technique (Higuchi, 1992, et al., Biotechnology 10: 413-417). By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundance of the specific mRNA from which the target sequence was derived can be determined for the respective tissues or cells. This direct proportionality between the concentration of the PCR products and the relative mRNA abundance is typically true in the linear range of the PCR reaction. The final concentration of the target DNA in the plateau portion of the curve is determined by the availability of reagents in the reaction mix and is independent of the original concentration of target DNA. In other embodiments, multiplexed, tandem PCR (MT-PCR) can be employed, which uses a two-step process for gene expression profiling from small quantities of RNA or DNA, as described for example in US Pat. Appl. Pub. No. 20070190540. In the first step, RNA is converted into cDNA and amplified using multiplexed gene specific primers. In the second step each individual gene is quantitated by real time PCR.

In some embodiments, target nucleic acid can be quantified using blotting techniques, which are well known to those of skill in the art. Southern blotting involves the use of DNA as a target, whereas Northern blotting involves the use of RNA as a target. Each provides different types of information, although cDNA blotting is analogous, in many aspects, to blotting or RNA species. Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (usually labelled) under conditions that promote denaturation and rehybridisation. Because the probe is designed to base pair with the target, the probe will bind a portion of the target sequence under renaturing conditions. Unbound probe is then removed, and detection is accomplished as described above. Following detection/quantification, one may compare the results seen in a given subject with a control reaction or a statistically significant reference group or population of control subjects as defined herein. In this way, it is possible to correlate the amount of a biomarker nucleic acid detected with the likelihood that a subject is at risk of cancer progression.

Also contemplated herein are biochip-based technologies such as those described by Hacia et al. (1996, Nature Genetics 14: 441-447) and Shoemaker et al. (1996, Nature Genetics 14: 450-456). Briefly, these techniques involve quantitative methods for analysing large numbers of genes rapidly and accurately. By tagging genes with oligonucleotides or using fixed probe arrays, one can employ biochip technology to segregate target molecules as high-density arrays and screen these molecules on the basis of hybridization. See also Pease et al. (1994, Proc. Natl. Acad. Sci. U.S.A. 91: 5022-5026); Fodor et al. (1991, Science 251: 767-773). Briefly, nucleic acid probes to the target sequence(s) are made and attached to biochips to be used in screening and diagnostic methods, as outlined herein. The nucleic acid probes attached to the biochip are designed to be substantially complementary to specific expressed target sequence(s), for example in sandwich assays, such that hybridization of the target sequence and the probes of the present invention occur. This complementarity need not be perfect; there may be any number of base pair mismatches, which will interfere with hybridization between the target sequence and the nucleic acid probes of the present invention. However, if the number of mismatches is so great that no hybridization can occur under even the least stringent of hybridization conditions, the sequence is not a complementary target sequence. In certain embodiments, more than one probe per sequence is used, with either overlapping probes or probes to different sections of the target being used. That is, two, three, four or more probes, with three being desirable, are used to build in a redundancy for a particular target. The probes can be overlapping (i.e. have some sequence in common), or separate.

In an illustrative biochip analysis, oligonucleotide probes on the biochip are exposed to or contacted with a nucleic acid sample suspected of containing one or more biomarker polynucleotides under conditions favouring specific hybridization.

Once isolated by the methods disclosed herein, the nucleic acid may be fragmented, for example, by sonication or by treatment with restriction endonucleases. Suitably, cDNA can be fragmented such that resultant DNA fragments are of a length greater than the length of the immobilized oligonucleotide probe(s) but small enough to allow rapid access thereto under suitable hybridization conditions. Alternatively, fragments of cDNA may be selected and amplified using a suitable nucleotide amplification technique, as described for example above, involving appropriate random or specific primers.

Other illustrative examples of methods by which nucleic acid can be amplified include Loop mediated isothermal amplification (LAMP), recombinase polymerase amplification (RPA), rolling circle amplification and primer generation-rolling circle amplification (PG-RCA).

In an embodiment, the target nucleic acid is amplified in a reaction vessel in the presence of the thermoplastic polymer substrate.

### Kits

In another aspect, there is provided a kit for isolating nucleic acid from a cell lysate, the kit comprising:

(a) a thermoplastic polymer substrate, as herein described;
(b) an elution buffer, as herein described; and
(c) optionally, a cell lysis buffer, as herein described;
wherein the thermoplastic polymer substrate has a net negative charge in solution.

In an embodiment, the kit may comprise one or more components and/or reagents and/or devices for use in performing the methods disclosed herein. The kits may contain component a and/or reagents for analyzing the expression of a target nucleic acid sequence in the nucleic acid recovered by the methods disclosed herein.

Kits for carrying out the methods of the present invention may also include, in suitable container means, (i) one or more reagents for detecting the one or more target nucleic acid sequences, (ii) one or more nucleic acid primers and/or probes that specifically bind to the target nucleic acid sequences, (iii) one or more probes that are capable of detecting and/or measuring the expression of the one or more target sequences (iv) one or more labels for detecting the presence of the probes and/or (iv) instructions for how to measure the level of expression of the one or more target sequences. Also included may be enzymes suitable for amplifying nucleic acids including various polymerases (Reverse Transcriptase, Taq, Sequenase™ DNA ligase etc. depending on the nucleic acid amplification technique employed), and deoxynucleotides and buffers to provide the necessary reaction mixture for amplification. Such kits may also comprise, in suitable means, distinct containers for each individual component and/or reagent, as well as for each primer and/or probe. The kit may also feature various devices (e.g., one or more) for performing any one of the methods described herein; and/or printed instructions for using the kit to detect and/or quantify the expression of one or more target nucleic acid sequences. The container means of the kits will generally include at least one vial, test tube, flask, bottle, syringe and/or other container into which one or more reagents will be placed or suitably aliquoted. Where a second and/or third and/or additional component is provided, the kit will also generally contain a second, third and/or other additional container into which this component may be placed. Alternatively, a container may contain a mixture of more than one reagent, as required. The kits may also include means for containing the one or more reagents (e.g., primers or probes) in close confinement for commercial sale. Such containers may include injection and/or blow-moulded plastic containers into which the desired vials are retained.

The kits may further comprise positive and negative controls, including a reference sample, as well as instructions for the use of kit components contained therein, in accordance with the methods disclosed herein.

The kits may also optionally include appropriate reagents for detection of labels, positive and negative controls, washing solutions, blotting membranes, microtiter plates, dilution buffers and the like.

In an embodiment, the kit comprises:

(a) a thermoplastic polymer substrate, as herein described;
(b) an elution buffer, as herein described; and
(c) a cell lysis buffer, as herein described;
wherein the thermoplastic polymer substrate has a net negative charge in solution.

In an embodiment, the thermoplastic polymer substrate is selected from the group consisting of a polyamide, polylactic acid, acrylonitrile butadiene styrene and alloys or composites comprising any of the foregoing. In an embodiment, the alloy or composite comprises polylactic acid. In an embodiment, the thermoplastic polymer substrate is an alloy comprising polylactic acid and a metal. In an embodiment, the metal is selected from the group consisting of copper and aluminium. In an embodiment, the thermoplastic polymer substrate is a composite comprising polylactic acid and carbon.

In an embodiment, the thermoplastic polymer substrate has an elongated structure with an average diameter from about 1 mm to about 3 mm. In an embodiment, the thermoplastic polymer substrate has an elongated structure with a length from about 1 to about 30 mm, preferably from about 10 mm to about 15 mm.

In an embodiment, the cell lysis buffer comprises a chaotropic salt. In an embodiment, the cell lysis buffer comprises a chaotropic salt in an amount that is from about 375 mM to about 6M, preferably in an amount that is about 1.5 M. In an embodiment, the chaotropic salt is guanidine chloride.

In an embodiment, the elution buffer is a PCR buffer.

In an embodiment, the kit further comprises instructions for using the components of the kit to isolate nucleic acid from a cell lysate in accordance with the methods herein described.

In another aspect, there is provided a thermoplastic polymer substrate, as herein described, for isolating nucleic acid from a cell lysate in accordance with the methods herein described, wherein the thermoplastic polymer substrate has a net negative charge when exposed to the cell lysate.

In another aspect, there is provided a thermoplastic polymer substrate, as herein described, when used for isolating nucleic acid from a cell lysate in accordance with the methods herein described, wherein the thermoplastic polymer substrate has a net negative charge when exposed to the cell lysate.

In another aspect, there is provided use of a thermoplastic polymer substrate, as herein described, in the manufacture of a device for isolating nucleic acid from a sample containing nucleic acid in accordance with the methods herein described, wherein the thermoplastic polymer substrate has a net negative charge in solution.

### EXAMPLES

### Materials and Methods

### A. 3D Printed “DipStix”

DipStix were 3D printed using fused deposition modeling methodology. 3D printing was primarily performed on an open source Makerfarm i3v (USA) platform with a 0.4 mm print nozzle, however propriety Up Plus 2 (China) and Up Mini (China) platforms were also used to successfully print DipStix. Slicing of the DipStix 3D model for 3D printing on the Maskerfarm i3v was performed using Simplify3D software (USA). 3D Printing parameters within Simplify 3D, including print speed, temperature, retraction settings, etc, were optimised for the different 3D filament materials so that a similar print quality was conserved between each material.

### B. General Nucleic Acid (NA) Isolation Protocols

Briefly, for characterization studies, 100 μL of chaotropic lysis buffer (50 mM Tris-HCl pH 8.0, 1.5 M guanidine-HCl, and 1% v/v Triton-X. Sigma Aldrich) containing 100 ng of purified HeLa cell gDNA (New England Biolabs) or 100 ng of purified BT474 cell gDNA and 10 Cpg BSA, to mimic a crude biological lysate. For complex samples, 100 μL lysis buffer was added to 50 μL cell suspensions (˜10⁶ cells), a single 5 mm leaf disc cutting, 100 uL whole blood or 100 μL milk. DipStix was then left in the crude lysate for 5 mins unless otherwise stated. This was followed by a wash step of five 1 sec dips into water. DipStix was then transferred to tubes containing either recombinase polymerase amplification (RPA) or quantitative polymerase chain reaction (qPCR) reactions for NA amplification. For RPA, the stick segment was broken off from the handle and left in the reaction. For qPCR, DipStix was incubated in 20 μL of 1×PCR buffer (2× GoTaq Clear, 5 mM MgCl₂, 0.1 mM dNTP, 4 μM SYTO-9 (50 μM), 0.05 U/μL Hot Start) and then removed because the opaque structure interfered with the fluorescence acquisition. 5 μL of 1×PCR buffer containing DNA polymerase, dNTP, primers and intercalating dye was subsequently added prior to thermocycling. DipStix printed with Bilby 3D natural PLA to the 2 mm diameter version was used in all experiment unless stated otherwise.

### C. DNA Detection

For isothermal RPA, the TwistAmp Basic kit (TwistDx) was used as recommended by the manufacturer with minor modifications. Briefly, RPA was performed at 37° C. for 15 mins in 12.5 μL reactions were used supplemented with 250 nM of each primer and 14 mM MgAc (magnesium acetate).

All qPCR experiments were perform on the ABI 7500 qPCR platform (Life Technologies). For qPCR quantification of DNA, the Kapa2G Robust HotStart Kit (Kapa Biosystems) was used as directed by the manufacturer with minor modifications. Briefly each 25 μL reaction included Buffer A (Buffer A is part of the the Kapa2G Robust HotStart Kit and comprises 1.5 mM MgCl₂; Sigma-Aldrich), 250 nM of each primer, 25 μg BSA (New England Biolabs) and 100 nM Syto9 (Life Technologies). The thermocycling protocol was 95° C. for 2 mins, 40 cycles of 95° C. for 15 s, 57° C. for 15 s, 72° C. for 30 s.

### D. RNA Detection

For mRNA and miRNA experiments, the miScript reverse transcription kit (Qiagen) was used to generate cDNA as directed by the manufacturer. The benefit of the miScript system was the generation of cDNA from all RNA species based on a polyA/oligo dT approach. Briefly, DipStix harbouring RNA from ZR75-1 breast cell lysates was dipped into 25 μL of 1× miScript HiFlex buffer for 5 mins and subsequently removed. Then 5 μL of 1× HiFlex buffer containing reverse transcriptase and the required nucleics was added to reaction. Reverse transcription was performed at 37° C. for 60 mins followed by 95° C. for 5 mins to inactivate. The cDNA was then diluted with 100 μL of RNase-free water and 0.5 μL was added to each qPCR experiment. Primers sequences for Her3 and Actin mRNA are provided in Table 1, below. For miRNAs, the miScript miR200a, miR15a and RNU6b assays were used.

TABLE 1

| | 5′-Forward-3′ | 5′-Reverse-3′ |
|---|---|---|
| BRAF | ATAGGTGATTTTGGTC | AGTAACTCAGCAGCAT |
| | TAGCTACTGT | CTCAGG |
| LINE1 | GTCAGGGAGTTCCCTT | GGACCCTCCTAGCCAG |
| | TCCGAGTCAAAGAA | GTGCAAGATATAAT |
| SAT2 | ATAGAATCGAATGGAA | ATTATTCCATTCCATT |
| | TTATCATCGAATGG | CCATTAGATGATTC |
| Actin | CTGGAACGGTGAAGGT | CGGCCACATTGTGAAC |
| | GACA | TTTG |
| HER3 | CTGATCACCGGCCTCA | GGAAGACATTGAGCTT |
| | AT | CTCTGG |
| Arabidopsis | YGACTCTCGGCAACGG | GCGTTCAAAGAYTCGA |
| | ATA | TGRTTC |
| Tomato | YGACTCTCGGCAACGG | GCGTTCAAAGAYTCGA |
| | ATA | TGRTTC |

TRIzol reagent (Invitrogen) based total RNA extraction was also used as recommended by the manufacturer as comparison to the DipStix method. Here, 100 ng of purified RNA was then used in the miScript system as described above.

### E. Fluorescence Imaging

Fluorescence and bright field imaging of the Cy5 labelled short oligonucleotides on the black PLA/carbon fibre DipStix was performed using a Nikon Eclipse Ni fluorescent microscope at 4× magnification.

F. Scanning electron microscopy

To prepare DipStix for scanning electron microscopy (SEM), samples were incubated at 45° C. overnight to remove any contaminates, and then affixed onto 25 mm SEM specimen mounts using 25 mm carbon conductive tabs and then further decontaminated using an Evactron Model 25 plasma cleaner (XIE Instruments). Edges of the DipStix samples were dagged with carbon paint to increase conductivity. The samples were then coated with 20 nm of platinum using a Baltek Med 020 sample coater and then stored in a vacuum desiccator until use. Just prior to analysis, the samples were decontaminated once more using Evactron Model 25 plasma cleaner. A Jeol JSM-7001F field emission scanning electron microscope (FE-SEM) was used to examine the surface details of the samples in secondary electron detection mode at 8 kV or 5 kV.

### G. Streaming Potential

Streaming potential measurements were performed in a solution of NaCl (1 mM) on thin films of 3D printed thermoplastic material using an Anton Paar SurPASS streaming potentiometer (Germany). pH titration was performed using a solution of NaOH (1 M). Measurements were analysed using Fairbrother-Mastin approach²⁶.

### Example 1: Isolation of Nucleic Acid Using Thermoplastic Material

Through serendipity, it was surprisingly found that 3D printing material can isolate DNA that was compatible with downstream amplification protocols. To ascertain whether this phenomenon was a common characteristic of other consumer 3D printing substrates, a simple tool was designed for convenient experimentation (FIG. 1A). This proof-of-concept 3D printed tool, referred to herein as “DipStix”, was designed to fit into a 0.2 mL tube and consisted of a 1.5 cm extension (stick) of 3 mm diameter attached to square holder. Briefly, DipStix was submerged into crude cell lysate solution, removed and washed by quickly by dipping it into water 5 times. The DipStix was then placed into a nucleic acid amplification reaction mixture. A feature of the DipStix tool was that one could easily break the narrow “stick” section off and seal the tip in the reaction tube, thereby reducing the risk of cross contamination during use. The DipStix design underscored the versatility of 3D printers for rapid prototyping and also for aiding research by facilitating equipment customization.

### Example 2: 3D Printing Thermoplastic Polymers as Substrates for Nucleic Acid Isolation

DipStix were printed with various 3D printing substrates to test for their effectiveness for nucleic acid isolation from a cell lysate prepared with common lysis buffers and for subsequent amplification (FIG. 1B). Materials tested in this study included various PLA-, acrylonitrile butadiene styrene (ABS)- and nylon-based thermoplastic polymer substrates.

The data in FIG. 1B show that all substrates tested were capable of isolating a sufficient amount of DNA from the cell lysate (containing about 1 ng/μL of DNA) for subsequent nucleic acid amplification by the isothermal RPA. RPA was chosen in this initial studies because of its potential for point-of-care (POC) applications. Hence, thermoplastic polymer substrates are an ideal companion to RPA and other isothermal systems-based applications for nucleic acid isolation and amplification.

Of the 10 materials tested, the wood-containing PLA substrate resulted in the poorest amplification efficiency. Whilst further studies are required, this result may have been attributed, at least in part, to residual phenolic compounds from wood that could have inhibited nucleic acid amplification by RPA. Lastly, since amplification occurred in all instances where DipStix was used and not from the DNA in chaotropic lysis buffer control, the data indicate that the method can remove inhibitors of nucleic acid amplification by RPA.

These data show that thermoplastic polymer substrates are suitable for laboratory-based NA isolation. In addition, since only a vessel of water for washing was required, the DipStix strategy is particularly suitable for POC applications.

For the purposes of brevity, the following studies were performed using the PLA substrate, “PLA Bilby 3D (natural)”, unless stated otherwise.

### Example 3: Low Sample Requirements

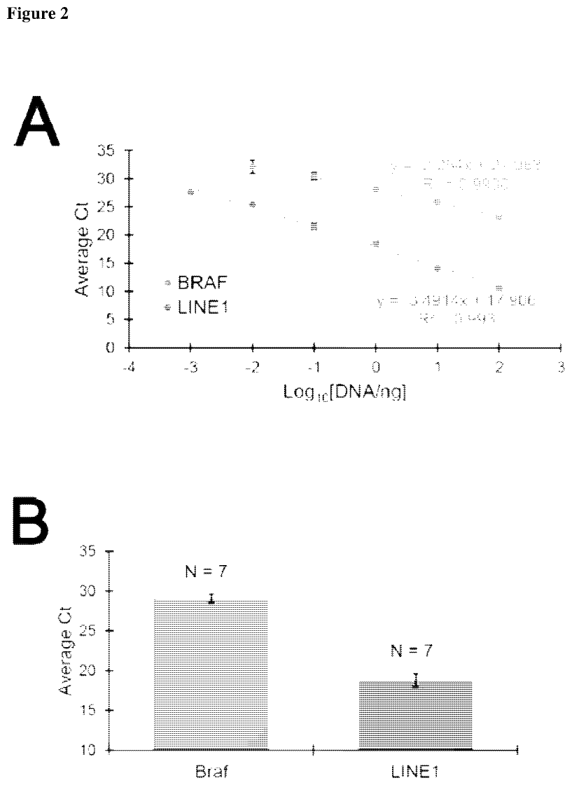

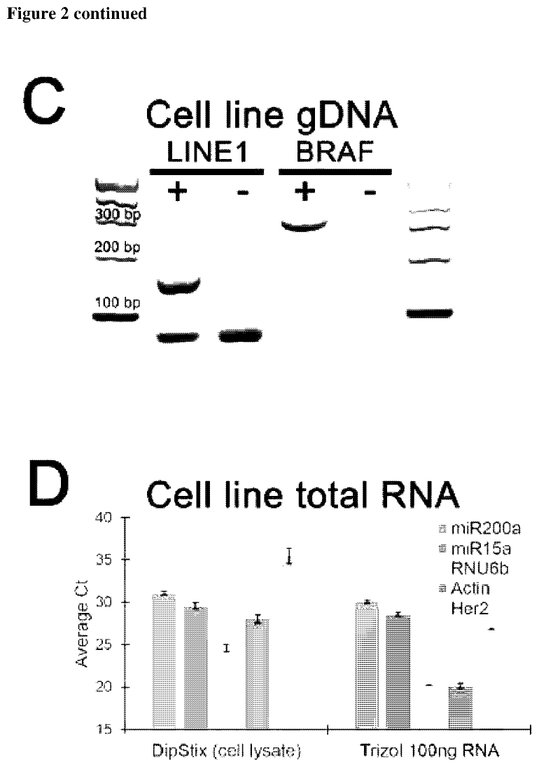

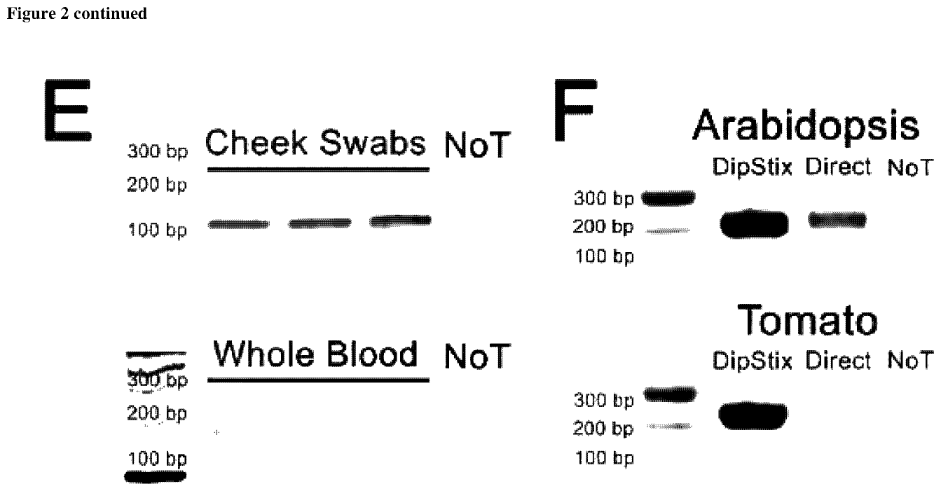

This experiment was conducted to determine the amount of sample required to obtain detectable amplification of NA. Briefly, qPCR was employed on DipStix-isolated DNA from 30 μL cell lysate material comprising 100 ng to 1 pg of cell line-derived DNA. A high copy gene (LINE1) and low copy gene (BRAF) were used in this study. As shown in FIG. 2A, as little as 1 pg of DNA was required to detect LINE1, while the limit of detection for BRAF was 10 pg. The data also suggest that the method removed a sufficient amount of any inhibitors of nucleic acid amplification from the cell lysate, such that one could have sensitivities approaching a single genome copy (assuming 1 human genome copy is approximately 3 pg). The amount of DNA isolated with DipStix was also consistent across 7 individual DipStix for a sample of known DNA concentration (see FIG. 2B), as indicated by the similar qPCR cycle threshold (Ct) values. This indicates that the method was highly reproducible (CV=3.10%, N=7).

### Example 4: Potential DipStix Applications

The DipStix method was extended to potential NA-based application reactions ranging from research to diagnostic applications to demonstrate its feasibility as a NA isolation tool. To this end, DNA (see FIG. 2C), mRNA and miRNA (see FIG. 2D) were successfully isolated and amplified from crude mammalian cell line lysates using RPA and qPCR, respectively. DNA was successfully isolated and detected from cheek swabs and whole blood with RPA (see FIG. 2E), and DNA was successfully isolated and detected from crude plant extracts with PCR (see FIG. 2F). These data demonstrate the potential for a range of NA applications and compatibility with various commonly used NA amplification protocols.

### Example 5: Rapid NA Capture and Release

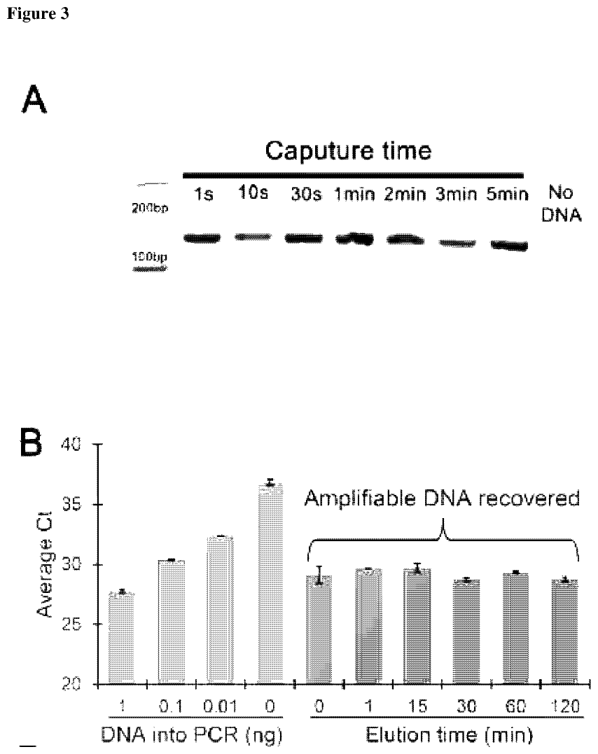

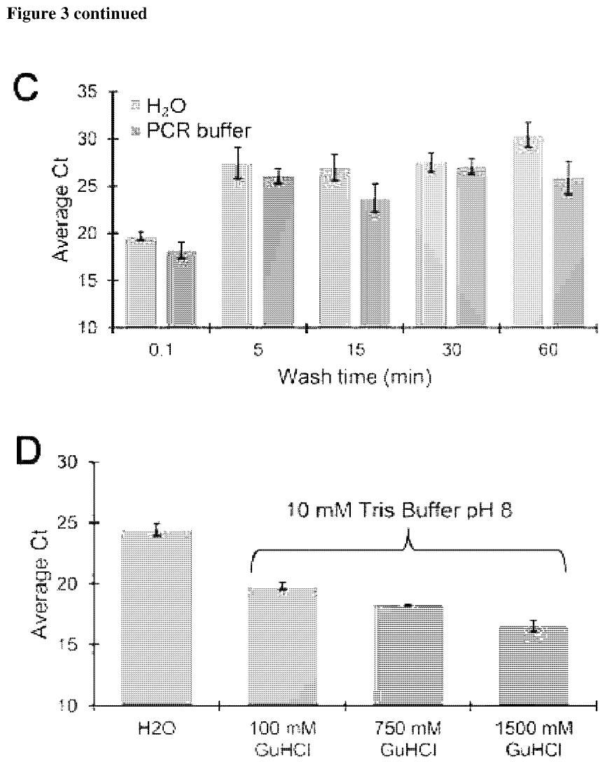

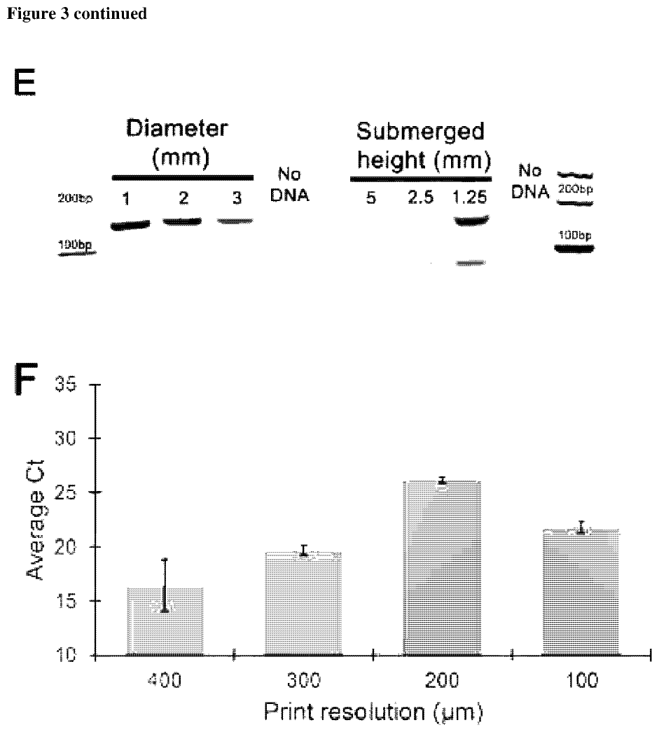

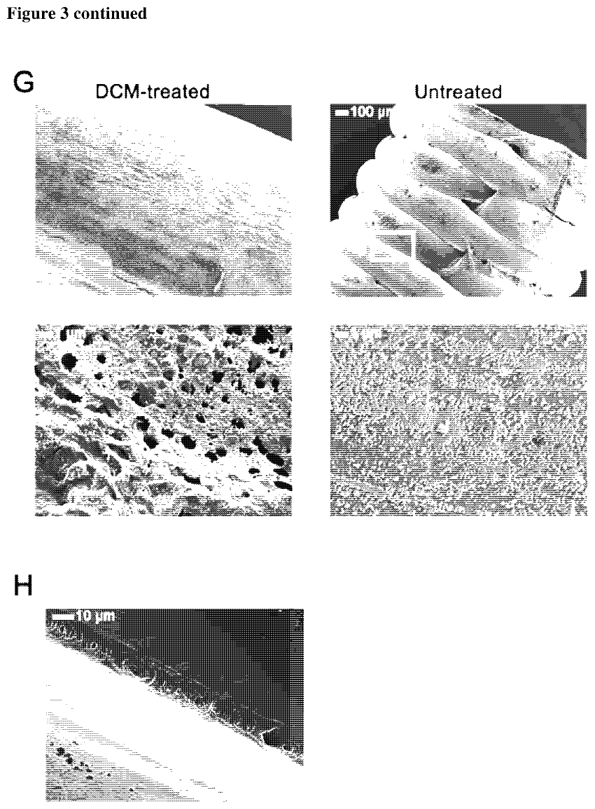

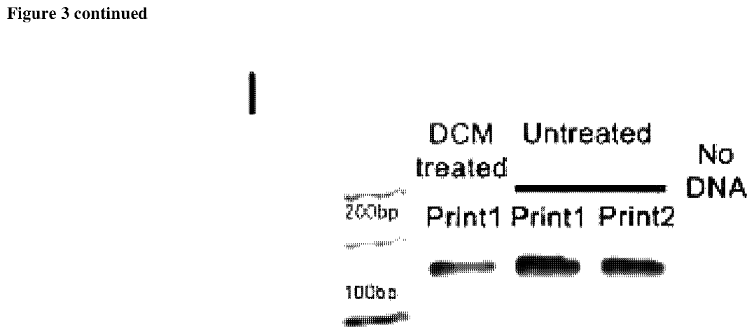

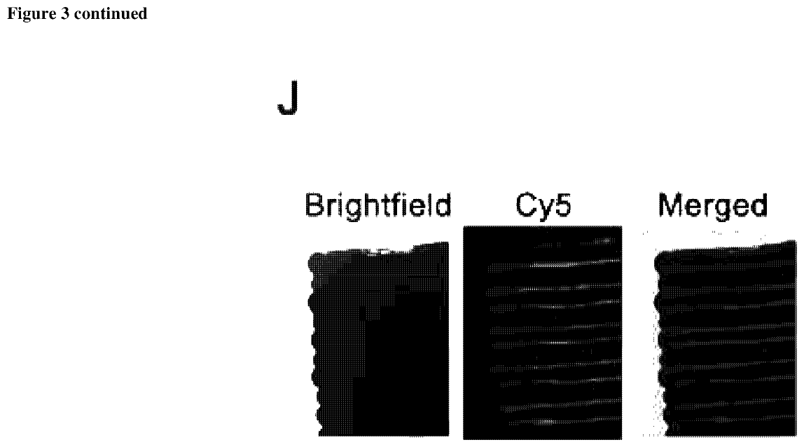

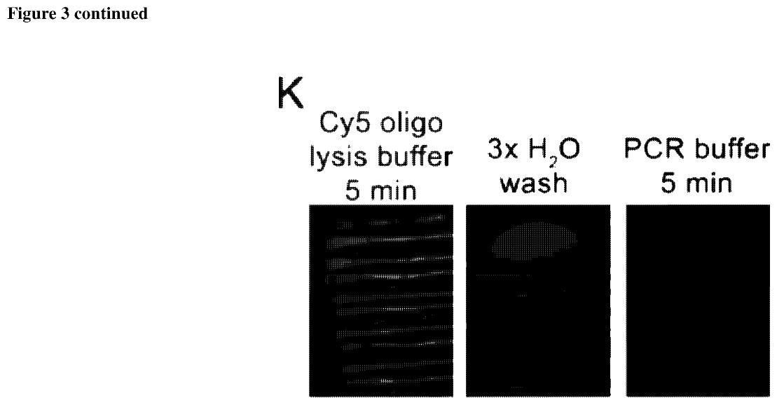

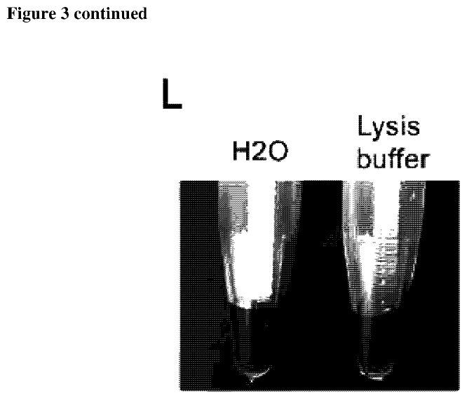

Experiments were conducted to ascertain the kinetics of DNA binding to the thermoplastic polymer substrate and subsequent release (see FIGS. 3A-C). Briefly, DipStix were exposed to the cell lysate material for a period of time ranging from a rapid immersion into the cell lysate (0 seconds) to an immersion for 5 minutes, followed by RPA. As shown in FIG. 3A, similar amplification yields were observed across all time points, suggesting that all amplifiable DNA bound rapidly to the thermoplastic polymer substrate, at least within the studied timescale.

Likewise, DNA could either release into the amplification buffer instantaneously or over a period of time and the amount of DNA released would be reflected by the yield of amplification. In this experiment, qPCR was used because RPA was not suitable for qualitative measurements over the intended study time frames. Briefly, DNA loaded DipStix were immersed into the qPCR buffer for a period of time ranging from a rapid immersion (0 seconds) to an immersion for 120 minutes to allow the DNA bound to the substrate to elute into the buffer. This was then followed by qPCR. DNA standards of known amounts were also included to estimate the amount of DNA available for PCR. The Ct values, which represented the amount of DNA present, were used to evaluate amplification yield. As shown in FIG. 3B, similar Ct values were observed across all time points, indicating that all amplifiable DNA was rapidly eluted from the thermoplastic polymer substrate, at least within studied timescale. The amount of amplifiable DNA released was estimated to be around 0.28±0.04 ng from a 1 ng/μL sample.

Consideration was then given to the wash step to ascertain whether or not the type of buffer (H₂O or PCR buffer) and the length of wash could affect subsequent NA amplification. As shown in FIG. 3C, washing the thermoplastic polymer substrate with either water or PCR buffer had minimal effect on subsequent NA amplification, as comparable Ct values were observed. However, between the 0.1 and >5 min wash lengths, an average increase of 7.8 Ct was observed, suggesting a rapid and substantial loss of DNA, consistent with the rapid desorption seen in FIG. 3B. In addition, the similar Ct values that were seen where greater than 5 minute washes were conducted suggest that an equilibrium between DNA loss and binding in buffer was reached. The data also indicated that, even after a 60 minute wash, sufficient DNA could still be recovered from the thermoplastic polymer substrate for subsequent amplification and detection.

### Example 6: DNA Binding Partly Electrostatically Driven

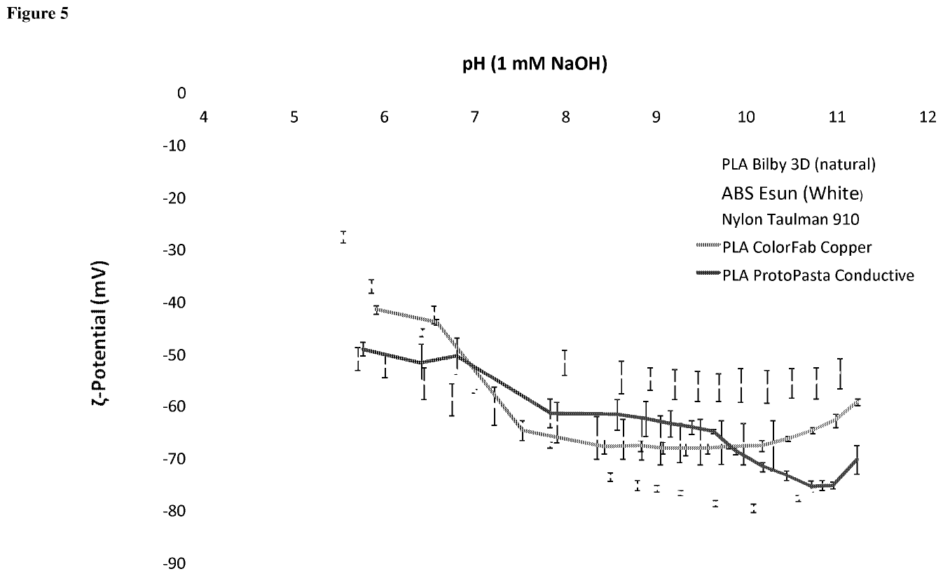

This experiment was conducted to ascertain whether the surface charge of the thermoplastic polymer substrate was attributing to its ability to isolate DNA from the cell lysate. A streaming potential measurement was made on the PLA-based thermoplastic polymer substrate. Surprisingly, it was found that the PLA-based thermoplastic polymer substrate was negatively charged (see FIG. 5), hence ruling out a direct surface charge interaction. This hypothesis was partially supported by the observation that DNA in water could bind to the DipStix and lead to positive PCR amplifications (see FIGS. 3C and D), which suggested a dependence, at least in part, on the physical structure of the thermoplastic polymer substrate. Furthermore, the amount of isolated DNA also increased (as indicated by lower Ct values) as the concentration of guanidine chloride (GuHCl) increased (see FIG. 3D), suggesting a salt-bridge/screening phenomena similar to the Boom method⁹ may be contributing, at least in part, to the isolation of DNA.

### Example 7: Nucleic Acid Isolation Effectiveness is a Function of Surface Area

Consideration was given as to whether or not the amount of NA bound to the surface of the thermoplastic polymer substrate was a function of the surface area of the substrate. This was assessed by manipulating the length and the diameter of the DipStix that was exposed to cell lysate material containing DNA. As shown in FIG. 3E, it was surprisingly found that, as the submerged length and the diameter decreased (i.e., as the surface area was reduced), the yield of RPA amplification increased celeris peribus. Increasing the print resolution up to 200 μm, which increased surface area, was also found to reduce amplification yields (see FIG. 3F). However, at a 100 μm 3D printing resolution, Ct values improved, suggesting an increase in amplification yield. Without being bound by theory or by a particular mode of application, the effective surface area at 100 μm resolution, being visually smoother at the microscopic level, may have been similar to that of the 300 μm resolution printed substrate.

These observations were counterintuitive, as one would have expected more DNA and therefore greater amplification yields with an increase in surface area. However, the reverse effect was observed. This observation could have been attributed, at least in part, to more residual inhibitors of NA amplification (e.g., chaotropic salts) being bound to a larger surface area, resulting in reduced efficiency of subsequent enzymatic amplification, despite the presence of more DNA bound to the substrate.

### Example 8: Nucleic Acid Isolation Effectiveness is Dependent on Macroscopic Roughness

Based on the print resolution data in Example 7, above, consideration was given as to whether or not altering the surface roughness of the thermoplastic polymer substrate would affect its ability to isolate NA from a cell lysate. Briefly, the PLA-based thermoplastic polymer substrate (PLA Bilby 3D, natural) was treated with the organic solvent dichloromethane (DCM) to smoothen the grooves that are otherwise produced by the layer-by-layer 3D printing (see FIG. 3G). Under scanning electron microscopy (SEM), the DCM-treated substrate was visually smoother (see FIG. 3G; left panel). However, under higher magnification, the DCM-treated substrate was visually porous, in contrast to the untreated substrate, which was microscopically smoother (see FIG. 3G, right panel). On closer inspection between the print layers (see FIG. 3H), pores ranging from 1-10 microns were observed. A subsequent comparison between the performance of DCM-treated and untreated substrates (see FIG. 3I) showed that the porous DCM-treated substrate (with higher surface area) performed marginally poorer than the untreated substrate, consistent with data shown in FIGS. 3E-F. Potential enzymatic inhibition by DCM was ruled out, since DCM-treated substrates were left under vacuum overnight to evaporate off any residual DCM. Hence, these data suggest that the macroscopic roughness introduced by layer-by-layer printing of the thermoplastic polymer substrate contributed to NA isolation.

The performance of the thermoplastic polymer substrates prepared by using two different models of 3D printers was also evaluated, but no significant differences were observed (see FIG. 3I).

### Example 9: Nucleic Acid Localized to the Grooves Between Print Layers

The interaction between DNA and the surface of the thermoplastic polymer substrates was visualised by placing the DipStix tip in a lysis buffer solution containing 10 μM of Cy5 fluorophore-labelled short oligonucleotides (see FIGS. 3J-K). The black PLA/carbon fibre thermoplastic polymer substrate was used for this experiment to avoid the effect of auto-fluorescence. Fluorescence was exclusively observed between the print layers, indicating that DNA localized preferentially between the grooves (see FIG. 3J).

As the fluorescent DNA-labelled DipStix was put through the NA isolation protocol, the DipStix was imaged: (1) immediately after removal from the lysis buffer; (2) immediately after the wash step in water; and (3) immediately after being submerged in PCR buffer for 5 mins (see FIG. 3K). As expected, strong fluorescence was seen at the tip of the DipStix immediately after removal from the DNA-containing cell lysate, indicating DNA bound to the substrate. The level of fluorescence reduced significantly after the wash step, indicating that excess DNA was removed. Upon closer inspection, remaining bound DNA following the wash step was found almost exclusively in the grooves between print layers of the thermoplastic polymer substrate. Finally, after leaving the DipStix in PCR buffer, minimal fluorescence was seen, indicating that almost all remaining DNA was eluted into the PCR buffer and made available for subsequent amplification. This was consistent with the qPCR data (see FIG. 3B) where almost all amplifiable DNA was eluted into the PCR buffer almost instantaneously.

A further study was undertaken to ascertain how the lysis buffer interacted with the DipStix. Using lysis buffer or water supplemented with food colouring, rapid wicking of the dyed lysis buffer up the DipStix shaft was observed (see FIG. 3L). In contrast, dyed water localized to the surface of the DipStix that was in contact with the solution. The rapid wicking of the dyed lysis buffer was likely due the presence of surfactant (1% Tween 20) in the lysis buffer, which could be contributing, at least in part, to enhancing NA capture.

### Example 10: Streaming Potential of Thermoplastic Polymer Substrates

This study was performed to measure the streaming (zeta; ζ) potential of several thermoplastic polymer substrates. The method was performed on 1 mm films of thermoplastic polymer substrates in a solution of 1 mM NaCl using an Anton Paar SurPASS streaming potentiometer (Germany). pH titration was performed using solutions of 1 M NaOH and the measurements were analysed using the Fairbrother-Mastin approach. The streaming potential data, as shown in FIG. 5, demonstrate that PLA-based substrates (PLA Bilby 3D (natural), PLA ColorFab Copper and PLA ProtoPasta Conductive), styrene-based substrates (ABS Esun (white)) and nylon-based substrates (Nylon Taulman 910) were all negatively charged across a wide range of pH values. The presented inventors observed that the salt concentration did not change the net charge of the thermoplastic polymer substrate from negative to positive. A change in salt concentration only altered affect the strength of the charge measurement (e.g., weaker negative to stronger negative, and vice versa). The inventors did not observe any positive charge on the substrates at the pH ranges tested: As outlined in the seminal paper by Kirby & Hasselbrink Jr (2014, Electrophoresis, 25(2), 187-202), the zeta potential (effective surface charge) of the thermoplastic polymer substrates tested are expected to retain a net negative charge at higher ionic strengths than would be present in the lysis buffers that were used in the experiments disclosed herein, as compared to the lower ionic strengths (1 mM NaCl) that were employed in this experiment. Without being bound by theory or a particular mode of application, this may be due to the monovalent ions (guanidine-HCl and Tris-HCl) not be expected to absorb to surfaces and, hence, not affecting the surface charge density of the thermoplastic polymer substrates. A factor that may affect the sign of the zeta potential is the pH of the solution, which would dictate the degree of protonation/deprotonation of the thermoplastic polymer. As the lysis buffers tested here ranges between pH 5 and pH 11 and all thermoplastic polymers produced negative zeta potential values across this pH range, it can be readily inferred that the zeta potentials of the thermoplastic polymer substrates in the lysis buffers of the earlier experiments disclosed herein also retained a net negative charge, due to ionic screening effects.

### Example 11: The Effect Lysis Buffer and Washes on NA Isolation and Subsequent PCR Amplification

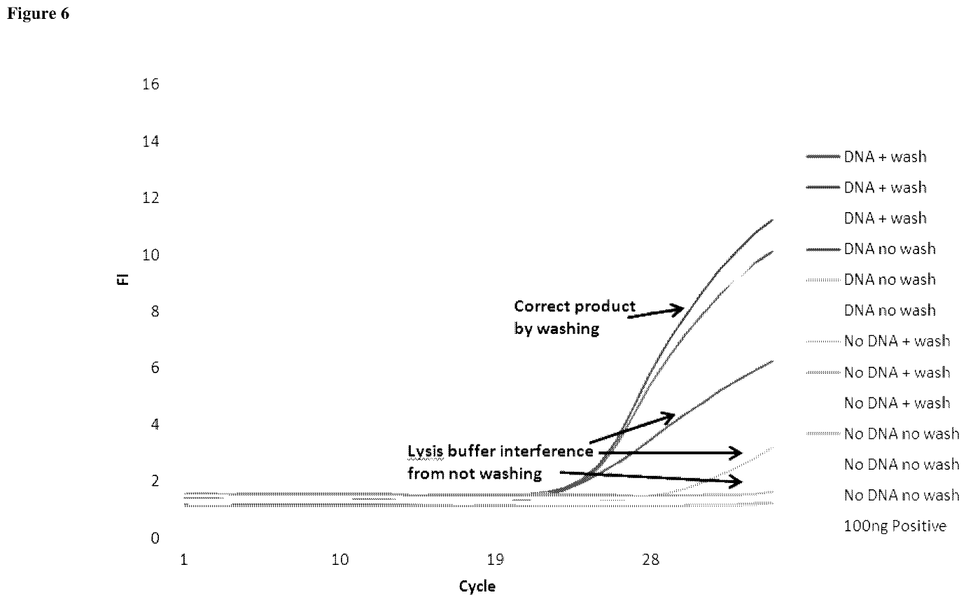

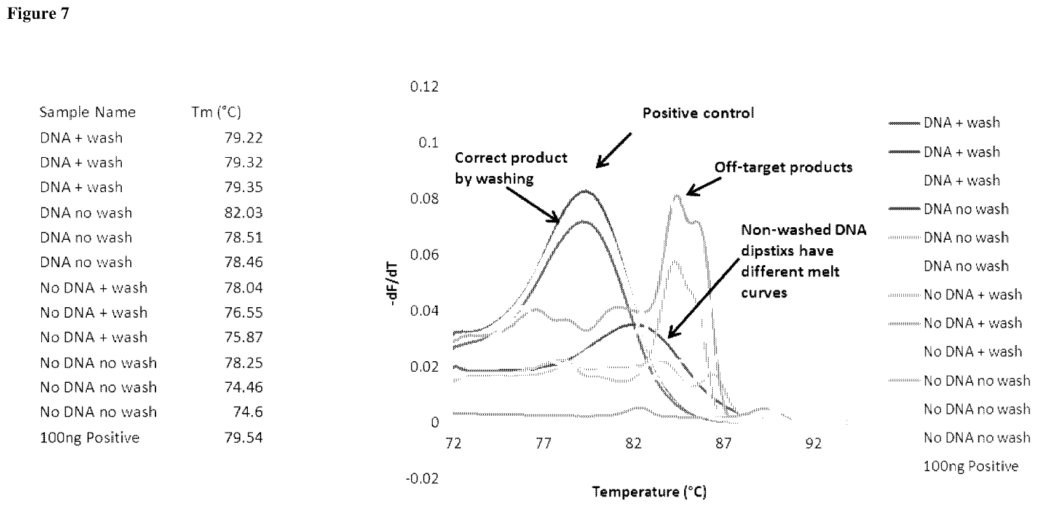

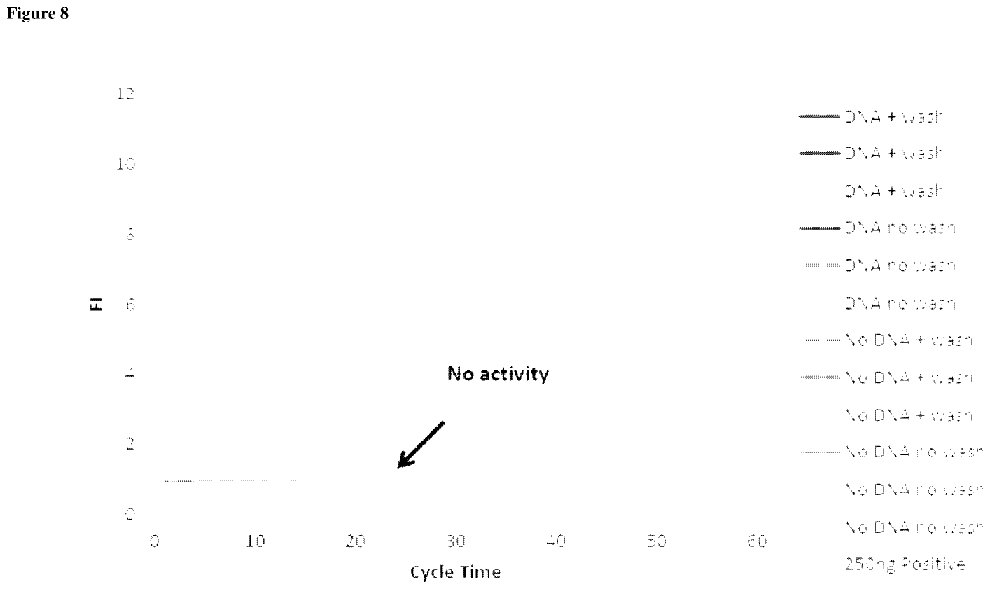

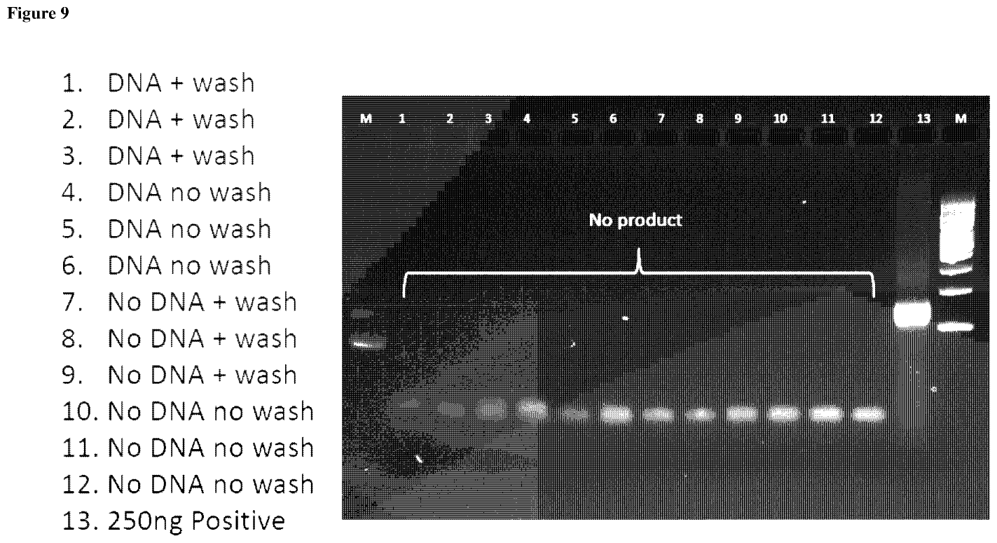

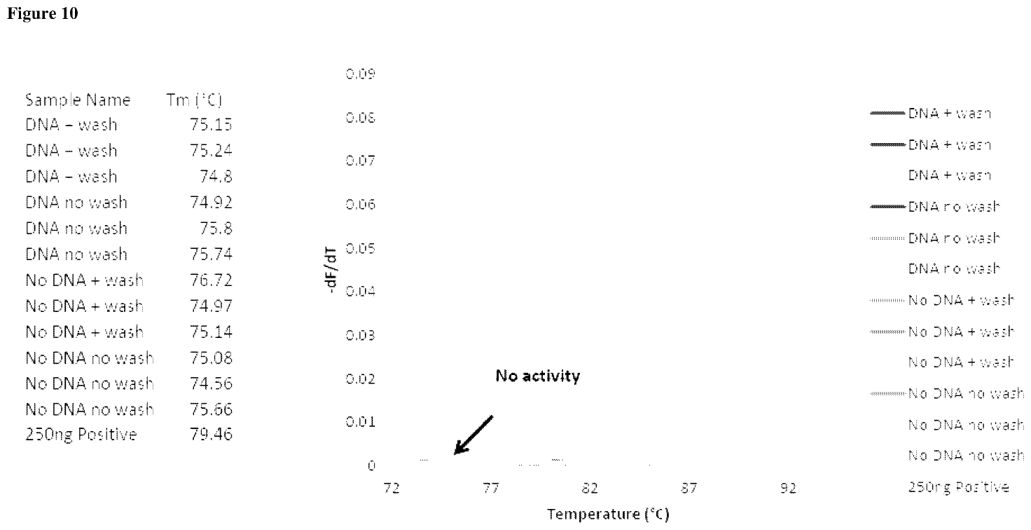

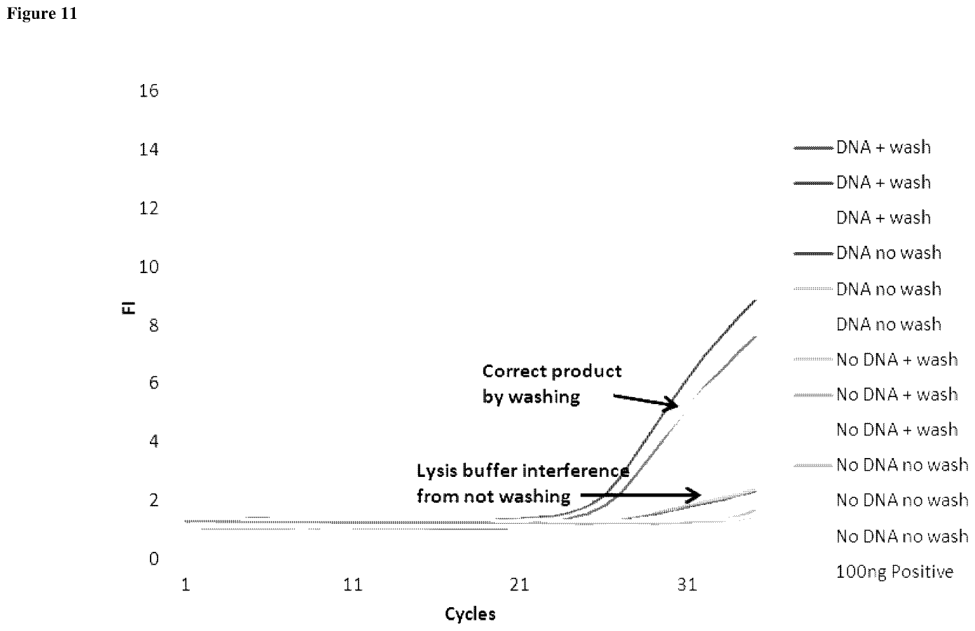

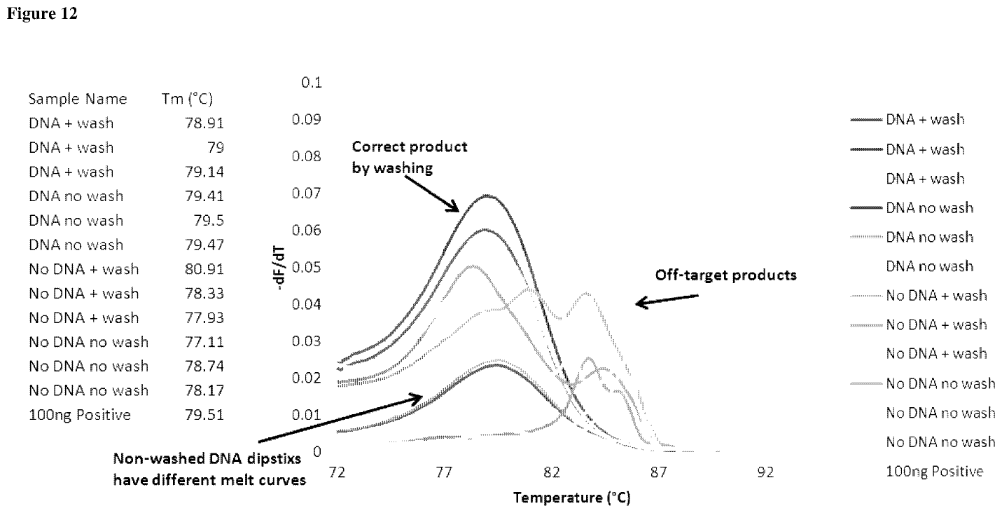

Experiments were performed to ascertain the effect of different lysis buffers on nucleic acid isolation using the thermoplastic polymer substrates and subsequent PCR amplification of the isolated DNA. This study was undertaken as certain lysis buffers are known to interfere with PCR amplification. Briefly, BT474 human breast cancer cells were lysed using three different lysis buffers: (i) a GuHCl-based lysis buffer comprising 50 mM Tris-HCl pH 8.0, 1.5 M guanidine-HCl, and 1% v/v Triton-X. Sigma Aldrich, (ii) SDS extraction buffer comprising 20 mM Tris-HCl, 1 mM EDTA, 0.5% (w/v) sodium dodecyl sulfate (SDS) and 800 units/mL Proteinase K and (iii) RIPA buffer comprising of 50 mM Tris-cl pH 7.4, 150 mM NaCl, 1 mM EDTA pH 8.0, 1% Triton X 100, 1% Sodium salt deoxycholate acid and 0.1% SDS. As shown in FIGS. 6-14, the GuHCl and RIPA lysis buffers had no discernible inhibitory effect on subsequent PCR amplification of target DNA, whereas SDS extraction buffer inhibited PCR amplification of target DNA.

For both GuHCl and RIPA lysis buffers, non-specific amplification of background products was evident, as shown by the presence of amplicons in the “no DNA” negative control samples. However, the data showed that the amplicons in the negative control samples only became evident 10 or more cycles after the amplicons generated from the positive control samples. Hence, a cut-off at less than 30 cycles could be used to exclude background amplification, yet still allow for the amplification of target nucleic acid.

### Example 12: The Effect of Salt Concentration on NA Isolation and Subsequent PCR Amplification

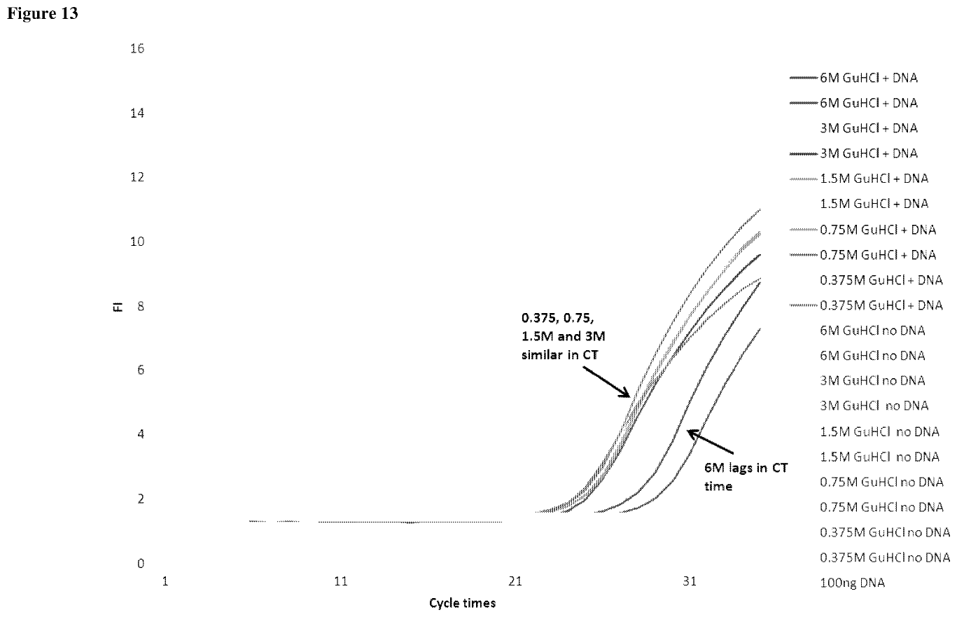

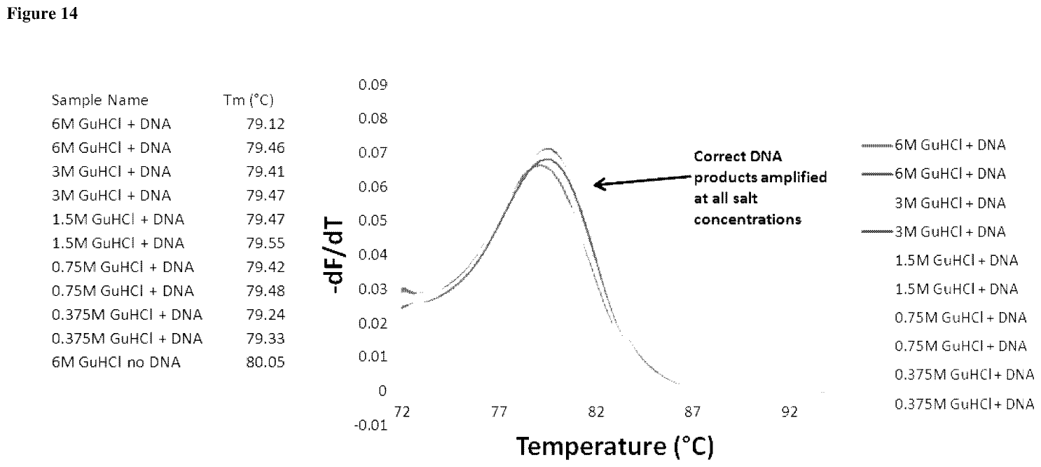

Experiments were performed to ascertain whether changes in salt concentration in the cell lysate has any effect on nucleic acid isolation and subsequent PCR amplification. As shown in FIGS. 13-14, lower salt concentrations worked better, noting that cycles times (CT) for DNA amplification decreased as the salt concentration in the lysis buffer was reduced. Higher salt concentrations (e.g., 6 M) still worked, although the CT values increased as a result. This increase in CT could negatively affect the lower limit of DNA amplification. It was noted that contaminants were found at above 30 CT. Moreover, lower salt concentrations were found to be optimal for DNA amplification as compared to higher salt concentrations, although amplicons were produced at all salt concentrations tested. The data also show that washing the thermoplastic polymer substrates with water after their removal from the lysis buffer reduced any inhibitory effect of the salt on qPCR amplification.

### DISCUSSION

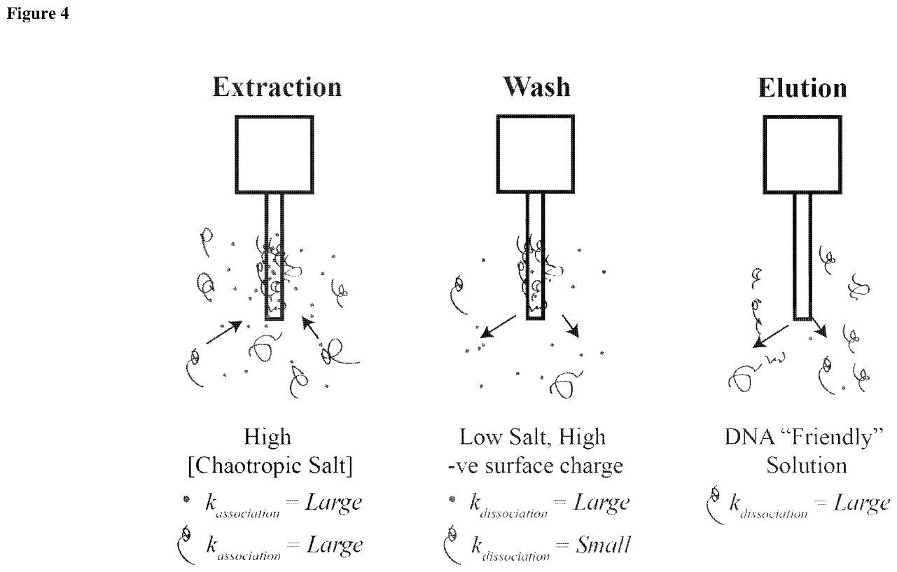

Without being bound by theory, or by a particular mode of application, the data from these studies suggest a likely mechanism of action contributing to nucleic acid (NA) isolation from cell lysate material using thermoplastic polymer substrates, as diagrammatically illustrated in FIG. 4. Under suitable salt conditions, NA and inhibitors of nucleic acid amplification rapidly bind to the surface of the thermoplastic polymer substrate (see FIG. 3A), including any grooves that may be formed between thermoplastic polymer substrate layers (see FIGS. 3J-K). The amount of NA bound to the thermoplastic polymer substrate is likely to could be enhanced by rapid wicking (see FIG. 3L) due to the presence of surfactants in the lysis buffer. Both NA and inhibitors of nucleic acid amplification appear to bind to the thermoplastic polymer substrate, as evidenced by the inverse relationship between surface area and amplification yields (see FIGS. 3E and F). Binding of NA onto the thermoplastic polymer substrate appears to occur almost instantly, as evidenced by the similar amplification yields over various exposure times (see FIG. 3A). During the low salt wash, excess NA and inhibitors of nucleic acid amplification are sufficiently removed to allow for amplification of NA (see FIGS. 1B and 3K). Furthermore, as NA appeared to preferentially localize to the grooves between layers of the thermoplastic polymer substrate of the DipStix (see FIGS. 3J and K), it is likely that some NA may be “trapped” within the pores of the thermoplastic polymer substrate (see FIGS. 3G and H), as evidenced by consistent recovery of DNA after extended periods of washing (see FIG. 3C). Finally, when the NA-bound thermoplastic polymer substrate is placed into a NA amplification buffer, which is conducive to NA solvation (elution), the remaining NA rapidly goes into solution (see FIGS. 3B, C and K) where it is available for NA amplification.

These data highlight a new approach to simple and rapid NA isolation by using thermoplastic polymer substrates, such as 3D printing polymer substrates. Due to the speed and simplicity of the methods disclosed herein, thermoplastic polymer substrates offer a favourable alternative to conventional NA isolation protocols. Moreover, with consumer level thermoplastic polymer substrates becoming more accessible, an easily customizable platform, such as the DipStix example disclosed herein, will find wider applications in both laboratory and POC-based applications where NA isolation is required.

### REFERENCES

1 Gregory, W. B., Jennifer, E. S.-W., Karteek, K. & James, F. R. 3D-printed bioanalytical devices. Nanotechnology 27, 284002 (2016).
2 Murphy, S. V. & Atala, A. 3D bioprinting of tissues and organs. Nat Biotech 32, 773-785, doi:10.1038/nbt.2958 (2014).
3 Ventola, C. L. Medical Applications for 3D Printing: Current and Projected Uses. Pharmacy and Therapeutics 39, 704-711 (2014).
4 Gross, B. C., Erkal, J. L., Lockwood, S. Y., Chen, C. & Spence, D. M. Evaluation of 3D Printing and Its Potential Impact on Biotechnology and the Chemical Sciences. Analytical chemistry 86, 3240-3253, doi: 10.1021/ac403397r (2014).
5 Berg, B. et al. Cellphone-Based Hand-Held Microplate Reader for Point-of-Care Testing of Enzyme-Linked Immunosorbent Assays. ACS Nano 9, 7857-7866, doi: 10.1021/acsnano.5b03203 (2015).
6 Patrick, W. G. et al. DNA Assembly in 3D Printed Fluidics. PLoS ONE 10, e0143636, doi:10.1371/journal.pone.0143636 (2016).
7 Chan, K. et al. Low-Cost 3D Printers Enable High-Quality and Automated Sample Preparation and Molecular Detection. PLoS ONE 11, e0158502, doi:10.1371/journal.pone.0158502 (2016).
8 Dineva, M. A., Mahilum-Tapay, L. & Lee, H. Sample preparation: a challenge in the development of point-of-care nucleic acid-based assays for resource-limited settings. Analyst 132, 1193-1199, doi: 10.1039/B705672A (2007).
9 Boom, R. et al. Rapid and simple method for purification of nucleic acids. Journal of clinical microbiology 28, 495-503 (1990).
Marko, M. A., Chipperfield, R. & Birnboim, H. C. A procedure for the large-scale isolation of highly purified plasmid DNA using alkaline extraction and binding to glass powder. Analytical biochemistry 121, 382-387 (1982).
11 DeAngelis, M. M., Wang, D. G. & Hawkins, T. L. Solid-phase reversible immobilization for the isolation of PCR products. Nucleic acids research 23, 4742-4743 (1995).
12 Wee, E. J., Lau, H. Y., Botella, J. R. & Trau, M. Re-purposing bridging flocculation for on-site, rapid, qualitative DNA detection in resource-poor settings. Chemical communications 51, 5828-5831, doi: 10.1039/c4ccl0068a (2015).
13 Hagan, K. A., Meier, W. L., Ferrance, J. P. & Landers, J. P. Chitosan-Coated Silica as a Solid Phase for RNA Purification in a Microfluidic Device. Analytical chemistry 81, 5249-5256, doi: 10.1021/ac900820z (2009).
14 Moeller, J. R., Moehn, N. R., Waller, D. M. & Givnish. T. J. Paramagnetic Cellulose DNA Isolation Improves DNA Yield and Quality Among Diverse Plant Taxa. Applications in Plant Sciences 2, 1400048, doi:10.3732/apps.1400048 (2014).
15 Su, X. & Comeau, A. M. Cellulose as a Matrix for Nucleic Acid Purification. Analytical biochemistry 267, 415-418, doi: http://dx.doi.org/10.1006/abio. 1998.2987 (1999).
16 Fisher, S. et al. A scalable, fully automated process for construction of sequence-ready human exome targeted capture libraries. Genome Biology 12, 1-15, doi:10.1186/gb-2011-12-1-r1 (2011).
17 Mosley, O. et al. Sample introduction interface for on-chip nucleic acid-based analysis of Helicobacter pylori from stool samples. Lab on a chip 16, 2108-2115, doi: 10.1039/c61c00228e (2016).
18 Lehmann, U., Vandevyver, C., Parashar, V. K. & Gijs, M. A. M. Droplet-Based DNA Purification in a Magnetic Lab-on-a-Chip. Angewandie Chemie International Edition 45, 3062-3067, doi: 10.1002/anie.200503624 (2006).
19 Sur, K. et al. Immiscible Phase Nucleic Acid Purification Eliminates PCR Inhibitors with a Single Pass of Paramagnetic Particles through a Hydrophobic Liquid. The Journal of Molecular Diagnostics: JMD 12, 620-628, doi: 10.2353/jmoldx.2010.090190 (2010).
20 Kim, J., Johnson, M., Hill, P. & Gale, B. K. Microfluidic sample preparation: cell lysis and nucleic acid purification. Integrative Biology 1, 574-586, doi: 10.1039/B905844C (2009).
21 Chen, W., Gerasimov, J. Y., Zhao, P., Liu, K. & Herrmann, A. High-Density Noncovalent Functionalization of DNA by Electrostatic Interactions. Journal of the American Chemical Society 137, 12884-12889, doi: 10.1021/jacs.5b05432 (2015).
22 Piepenburg, O., Williams, C. H., Stemple, D. L. & Armes, N. A. DNA Detection Using Recombination Proteins. PLoS Biol 4, e204, doi:10.1371/journal.pbio.0040204 (2006).
23 Lau, H. Y., Wang, Y., Wee, E. J. H., Botella, J. R. & Trau, M. Field Demonstration of a Multiplexed Point-of-care Diagnostic Platform for Plant Pathogens. Analytical chemistry, doi:10.1021/acs.analchem.6b01551 (2016).
24 Daher, R. K., Stewart, G., Boissinot, M. & Bergeron, M. G. Recombinase Polymerase Amplification for Diagnostic Applications. Clinical Chemistry 62, 947-958, doi: 10.1373/clinchem.2015.245829 (2016).
25 Couch, J. A. & Fritz, P. J. Isolation of DNA from plants high in polyphenolics. Plant Molecular Biology Reporter 8, 8-12, doi:10.1007/bf02668875 (1990).
26. Fairbrother & Mastin. J. Chem. Soc., Trans., 125, 2319-2330 (1924)

1. A method of isolating nucleic acid from a sample containing nucleic acid, the method comprising:
(a) exposing the sample to a thermoplastic polymer substrate under conditions that allow nucleic acid in the sample to reversibly bind to the substrate;
(b) washing the nucleic acid-bound substrate of (a) under conditions that preferentially remove non-nucleic acid impurities bound to the substrate; and
(c) exposing the washed nucleic acid-bound substrate of (b) to an elution buffer, thereby recovering the nucleic acid from the substrate;
wherein the thermoplastic polymer substrate has a net negative charge in solution.

2. The method of claim 1, wherein the thermoplastic polymer substrate is selected from the group consisting of a polyamide, polylactic acid, acrylonitrile butadiene styrene, and composites or alloys of any of the foregoing.

3. The method of claim 2, wherein the composite or alloy comprises polylactic acid.

4. The method of claim 3, wherein the thermoplastic polymer substrate is an alloy comprising polylactic acid and a metal.

5. The method of claim 4, wherein the metal is selected from the group consisting of copper and aluminium.

6. The method of claim 3, wherein the thermoplastic polymer substrate is a composite comprising polylactic acid and carbon.

7. The method of claim 1, wherein the sample is a biological sample.

8. The method of claim 7, wherein the biological sample is selected from the group consisting of blood, serum, plasma, urine, semen, amniotic fluid and spinal fluid.

9. The method of claim 7, wherein the biological sample is a cell lysate.

10. The method of claim 1, wherein the sample comprises a chaotropic salt.

11. The method of claim 10, wherein the sample comprises a chaotropic salt in an amount that is from about 375 mM to about 6M.

12. The method of claim 11, wherein the cell lysate of (a) comprises a chaotropic salt in an amount that is about 1.5 M.

13. The method of claim 10, wherein the chaotropic salt is guanidine chloride.

14. The method of claim 1, wherein the thermoplastic polymer substrate has an elongated structure with an average diameter from about 1 mm to about 3 mm.

15. The method of claim 1, wherein the thermoplastic polymer substrate has an elongated structure with a length from about 1 to about 30 mm.

16. The method of claim 15, wherein the thermoplastic polymer substrate has a length from about 10 mm to about 15 mm.

17. The method of claim 1, wherein step (a) comprises exposing the sample to the thermoplastic polymer substrate for a period of time from about 0.5 seconds to about 5 minutes.

18. The method of claim 17, wherein step (a) comprises exposing the sample to the thermoplastic polymer substrate for a period of time from about 0.5 seconds to about 1 minute.

19. The method of claim 17, wherein step (a) comprises exposing the sample to the thermoplastic polymer substrate for a period of time from about 0.5 seconds to about 30 seconds.

20. The method of claim 17, wherein step (a) comprises exposing the sample to the thermoplastic polymer substrate for a period of time from about 0.5 seconds to about 1 second.

21. The method of claim 17, wherein exposing the sample to the thermoplastic polymer substrate comprises dipping the thermoplastic polymer substrate into the sample.

22. The method of claim 1, wherein step (b) comprises washing the nucleic acid-bound substrate in water.

23. The method of claim 22, wherein step (b) comprises washing the nucleic acid-bound substrate in water for a period from about 5 seconds to about 1 minute.

24. The method of claim 22, wherein step (b) comprises dipping the nucleic acid-bound substrate into the water.

25. The method of claim 1, wherein the elution buffer is a PCR buffer.

26. The method of claim 1, wherein step (c) comprises exposing the washed nucleic acid-bound substrate to the elution buffer for a period of time from about 0.5 seconds to about 5 minutes.

27. The method of claim 26, wherein step (c) comprises exposing the washed nucleic acid-bound substrate to the elution buffer for a period of time from about 0.5 seconds to about 1 minute.

28. The method of claim 26, wherein step (c) comprises exposing the washed nucleic acid-bound substrate to the elution buffer for a period of time from about 0.5 seconds to about 1 second.

29. The method of claim 1, wherein exposing the washed nucleic acid-bound substrate to the elution buffer cell comprises dipping the washed nucleic acid-bound substrate into the elution buffer.

30. The method of claim 1, further comprising amplifying a target nucleic acid sequence from the nucleic acid recovered in step (c).

31. The method of claim 30, wherein the target nucleic acid is amplified in a reaction vessel in the presence of the thermoplastic polymer substrate.

32. A composition comprising nucleic acid recovered by the method of claim 1.

33. A kit for isolating nucleic acid from a cell lysate, the kit comprising:
(a) a thermoplastic polymer substrate;
(b) an elution buffer; and
(c) optionally, a cell lysis buffer;
wherein the thermoplastic polymer substrate has a net negative charge in solution.

34. The kit of claim 33, wherein the thermoplastic polymer substrate is selected from the group consisting of a polyamide, polylactic acid, acrylonitrile butadiene styrene and composites or alloys of any of the foregoing.

35. The kit of claim 34, wherein the composite or alloy comprises polylactic acid.

36. The kit of claim 35, wherein the thermoplastic polymer substrate is an alloy comprising polylactic acid and a metal.

37. The kit of claim 36, wherein the metal is selected from the group consisting of copper and aluminium.

38. The kit of claim 35, wherein the thermoplastic polymer substrate is a composite comprising polylactic acid and carbon.

39. The kit of claim 33, wherein the kit comprises a cell lysis buffer.

40. The kit of claim 39, wherein the cell lysis buffer comprises a chaotropic salt.

41. The kit of claim 40, wherein the cell lysis buffer comprises a chaotropic salt in an amount that is from about 375 mM to about 6M.

42. The kit of claim 41, wherein the cell lysis buffer comprises a chaotropic salt in an amount that is about 1.5 M.

43. The kit of claim 39, wherein the chaotropic salt is guanidine chloride.

44. The kit of claim 33, wherein the thermoplastic polymer substrate has an elongated structure with an average diameter from about 1 mm to about 3 mm.

45. The kit of claim 33, wherein the thermoplastic polymer substrate has an elongated structure with a length from about 1 to about 30 mm.

46. The kit of claim 45, wherein the thermoplastic polymer substrate has a length from about 10 mm to about 15 mm.

47. The kit of claim 33, wherein the elution buffer is a PCR buffer.

48. A thermoplastic polymer substrate for isolating nucleic acid from a sample containing nucleic acid in accordance with the method of claim 1, wherein the thermoplastic polymer substrate has a net negative charge in solution.

49. The thermoplastic polymer substrate of claim 48, wherein the thermoplastic polymer substrate is selected from the group consisting of a polyamide, polylactic acid, acrylonitrile butadiene styrene and composites or alloys of any of the foregoing.

50. The thermoplastic polymer substrate of claim 49, wherein the composite or alloy comprises polylactic acid.

51. The thermoplastic polymer substrate of claim 50, wherein the thermoplastic polymer substrate is an alloy comprising polylactic acid and a metal.

52. The thermoplastic polymer substrate of claim 51, wherein the metal is selected from the group consisting of copper and aluminium.

53. The substrate of claim 49, wherein the thermoplastic polymer substrate is a composite comprising polylactic acid and carbon.
